(19) United States
(12) Patent Application Publication
(10) Pub. No.: US 2025/0134418 A1
(43) Pub. Date: May 1, 2025

(54) ANALYTE SENSOR SYSTEM WITH ACTIVE AND PASSIVE ANTENNAS

(71) Applicant: Dexcom, Inc., San Diego, CA (US)

(72) Inventor: Ezdeen ELGHANNAI, Seattle, WA (US)

(21) Appl. No.: 18/917,685

(22) Filed: Oct. 16, 2024

Related U.S. Application Data
(60) Provisional application No. 63/594,475, filed on Oct. 31, 2023.

(51) Int. Cl. A61B 5/145

(52) U.S. Cl. CPC A61B 5/14532; A61B 2560/045; A61B 2560/0462

(57) ABSTRACT

Aspects of the present disclosure provide techniques for avoiding and/or reducing blocking of signals transmitted between an analyte sensor system and a display device. The analyte sensor system may include an analyte sensor configured to generate analyte data associated with analyte levels of a user. The system may include an antenna system having at least a first antenna and a second antenna. The first antenna is configured to transmit a first signal including at least the analyte data, and receive from a display device, a second signal including operational instructions. The second antenna is configured to receive the first signal from the first antenna and re-radiate the first signal towards the display device, and receive the second signal from the display device and re-radiate the second signal towards the first antenna. The system may include a circuit board configured to operatively connect the analyte sensor with the first antenna of the antenna system.

### CROSS-REFERENCE TO RELATED APPLICATIONS

This application claims priority to and benefit of U.S. Provisional Patent Application No. 63/594,475, filed Oct. 31, 2023, which is hereby assigned to the assignee hereof and hereby expressly incorporated by reference herein in its entirety as if fully set forth below and for all applicable purposes.

### TECHNICAL FIELD

The present disclosure relates generally to an electronic device, such as an analyte sensor system for monitoring analyte values of a user.

### BACKGROUND

Diabetes is a metabolic condition relating to the production or use of insulin by the body. Insulin is a hormone that allows the body to use glucose for energy, or store glucose as fat. When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher-than-normal blood sugar level is called “hyperglycemia.” Chronic hyperglycemia can lead to several of health problems, such as cardiovascular disease, cataract and other eye problems, nerve damage (neuropathy), and kidney damage. Hyperglycemia can also lead to acute problems, such as diabetic ketoacidosis-a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

A diabetes patient can receive insulin to manage blood glucose levels. Insulin can be received, for example, through a manual injection with a needle. Wearable insulin pumps are also available. Diet and exercise also affect blood glucose levels.

Diabetes conditions are sometimes referred to as “Type 1” and “Type 2”. A Type 1 diabetes patient is typically able to use insulin when it is present, but the body is unable to produce adequate insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

### SUMMARY

Aspects of the present disclosure provide an analyte sensor system. The analyte sensor system may include an analyte sensor configured to generate analyte data associated with analyte levels of a user of the analyte sensor system. The analyte sensor system may also include an antenna system having at least a first antenna and a second antenna. The first antenna is configured to transmit, to a display device, a first signal including at least the analyte data and receive, from the display device, a second signal including operational instructions. The second antenna is configured to receive the first signal from the first antenna and re-radiate the first signal towards the display device and receive the second signal from the display device and re-radiate the second signal towards the first antenna. The analyte sensor system may also include a circuit board configured to operatively connect the analyte sensor with the first antenna of the antenna system.

Aspects of the present disclosure also provide an antenna system for communicating analyte data. The antenna system may include a first antenna operatively coupled to an analyte sensor via a circuit board. The first antenna may be configured to transmit, to a display device, a first signal including at least the analyte data and to receive, from the display device, a second signal including operational instructions. The antenna system may also include a second antenna configured to receive the first signal from the first antenna and re-radiate the first signal towards the display device and to receive the second signal from the display device and re-radiate the second signal towards the first antenna.

Aspects of the present disclosure also provide an analyte monitoring system. The analyte monitoring system may include a display device and an analyte sensor system. The analyte sensor system may include an analyte sensor configured to generate analyte data associated with analyte levels of a user of the analyte sensor system, a first antenna, and a second antenna. The first antenna may be configured to transmit, to the display device, a first signal including at least the analyte data and to receive, from the display device, a second signal including operational instructions. The second antenna may be configured to receive the first signal from the first antenna and re-radiate the first signal towards the display device and to receive the second signal from the display device and re-radiate the second signal towards the first antenna. The analyte sensor system may also include a circuit board configured to operatively connect the analyte sensor with the first antenna. In some embodiments, the display device is configured to display the analyte data received from the first antenna of the analyte sensor system to the user.

### BRIEF DESCRIPTION OF THE DRAWINGS

Further aspects of the present disclosure will be more readily appreciated upon review of the detailed description of the various disclosed embodiments, described below, when taken in conjunction with the accompanying figures.

FIG. 1 illustrates aspects of an example system that may be used in connection with some embodiments.

FIG. 2 illustrates aspects of an example system that may be used in connection with some embodiments.

FIG. 3A is an example analyte sensor system, in accordance with some embodiments.

FIG. 3B is an example analyte sensor system, in accordance with some embodiments.

FIG. 4 illustrates aspects of an example analyte sensor system, in accordance with some embodiments.

FIG. 5 illustrates aspects of an example analyte sensor system, in accordance with some embodiments.

FIG. 6 illustrates aspects of an example analyte sensor system having a main antenna, in accordance with some embodiments.

FIG. 7 illustrates aspects of an example analyte sensor system having a first antenna and a second antenna, in accordance with some embodiments.

FIG. 8 illustrates an example incident wave and a reflected wave, in accordance with some embodiments.

FIG. 9 depicts a method for wireless communication by an analyte sensor system, according to some embodiments disclosed herein.

FIG. 10 depicts a method for communication between an analyte sensor system

and a display device in an analyte monitoring system, according to some embodiments disclosed herein.

FIG. 11 depicts aspects of an example health monitoring device, according to some embodiments disclosed herein.

FIG. 12 depicts aspects of an example health monitoring device, according to some embodiments disclosed herein.

The figures, described in greater detail in the description and examples below, are provided for purposes of illustration only, and merely depict typical or example embodiments of the disclosure. The figures are not intended to be exhaustive or to limit the disclosure to the precise form disclosed. It should also be understood that the disclosure may be practiced with modification or alteration, and that the disclosure may be limited only by the claims and the equivalents thereof.

### DETAILED DESCRIPTION

Aspects of the present disclosure relate to passive reflective antennas to boost multi-path wireless channel communications between an analyte sensor system and a display device or other receiver. An analyte sensor system may be configured to measure analyte data of a user and send the analyte data to various external devices, such as a display device (e.g., a smartphone or display) using an active, or main antenna. The analyte sensor system may also be configured to receive control information or other types of information from the display device using the active, or main antenna. In some embodiments, information exchanged between the analyte sensor system and display device may, for example, be transmitted and/or received over various types of communications protocols, such as BLUETOOTH, BLUETOOTH Low Energy (BLE), Wi-Fi, and combinations of the same and like.

Designing antennas for analyte sensor systems pose many challenges, such as, for example, shadowed and/or blocked signals between the display device and the analyte sensor system. Shadowing is the effect of received signal power fluctuations due to obstructions between a transmitter and receiver. Shadowing and/or blocking may be problematic in single antenna designs as a single antenna is more susceptible to being blocked and/or shadowed by various sources. This may lead to dropped data packets, poor signal reception, and the like between the analyte sensor system and the display device. This leads to an increase in retransmissions of data packets which increases power consumption. This may be problematic if the analyte sensor system is power-limited. One manner to help avoid issues of shadowed and/or blocked signals may be to design an analyte sensor system with multiple active antennas. However, having multiple active antennas may increase the complexity and cost associated with manufacturing the analyte sensor system, and potentially decrease battery life of the analyte sensor system by constantly retransmitting data and/or keeping a connection active.

As such, the present disclosure describes techniques for avoiding and/or reducing the issues described above associated with the shadowing and/or blocking of signals transmitted between an analyte sensor system and a display device. In some embodiments, in addition to using a main antenna to communicate signals between the analyte sensor system and display device, the analyte sensor system may include a passive antenna. For example, the passive antenna may be configured to passively receive signals from the display device and reflect or re-radiate these signals towards the main antenna of the analyte sensor system, allowing the main antenna of the analyte sensor system to still receive signals from the display device even though signal paths associated with the signals from the display device may be blocked to the main antenna by a body of a user of the analyte sensor system or some other obstruction.

The details of some example embodiments of the systems, methods, and devices of the present disclosure are set forth in this description and in some cases, in other portions of the disclosure. Other features, objects, and advantages of the disclosure will be apparent to one of skill in the art upon examination of the present disclosure, description, figures, examples, and claims. It is intended that all such additional systems, methods, devices, features, and advantages be included within this description (whether explicitly or by reference), be within the scope of the present disclosure, and be protected by one or more of the accompanying claims.

### System Overview and Example Configurations

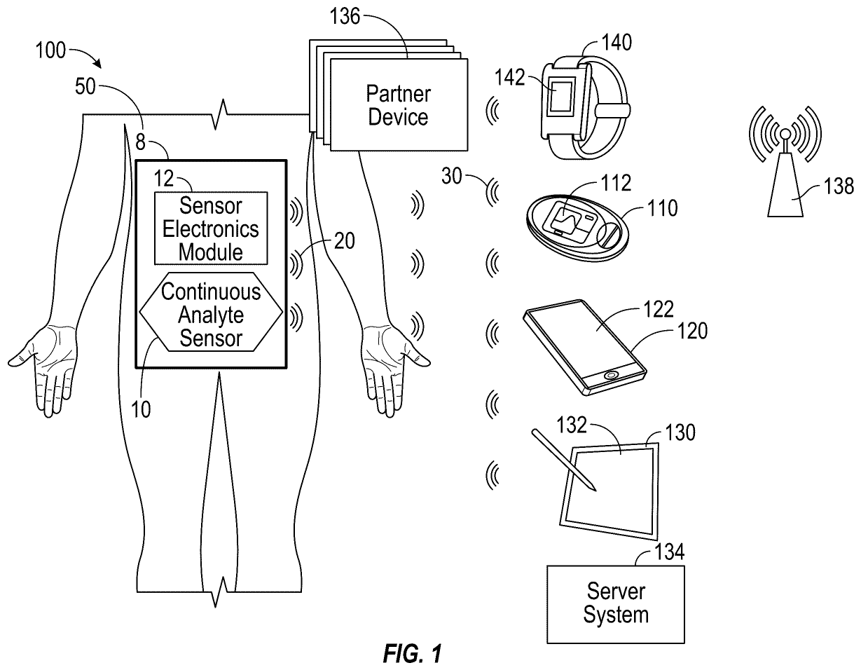

FIG. 1 depicts an analyte monitoring system 100 that may be used in connection with embodiments of the present disclosure that involve gathering, monitoring, and/or providing information regarding analyte values present in a user's body, including for example the user's blood glucose values, other analytes, multiple multiplexed or simultaneous measured analytes, or the like. System 100 depicts aspects of analyte sensor system 8 that may be communicatively coupled to display devices 110, 120, 130, and 140, partner devices 136, and/or server system 134.

Analyte sensor system 8 in the illustrated embodiment includes analyte sensor electronics module 12 and analyte sensor 10 associated with analyte sensor electronics module 12. Analyte sensor electronics module 12 may be electrically and mechanically coupled to analyte sensor 10 before analyte sensor 10 is implanted in a user or host. Accordingly, analyte sensor 10 may not require a user to couple analyte sensor electronics module 12 to analyte sensor 10. For example, analyte sensor electronics module 12 may be physically/mechanically and electrically coupled to analyte sensor 10 during manufacturing, and this physical/mechanical and electrical connection may be maintained during shipping, storage, insertion, use, and removal of analyte sensor system 8. As such, the electro-mechanically connected components (e.g., analyte sensor 10 and analyte sensor electronics module 12) of analyte sensor system 8 may be referred to as a “pre-connected” system. Analyte sensor electronics module 12 may be in wireless communication (e.g., directly or indirectly) with one or more of display devices 110, 120, 130, and 140. In addition, or alternatively to display devices 110, 120, 130, and 140, analyte sensor electronics module 12 may be in wireless communication (e.g., directly or indirectly) with partner devices 136 and/or server system 134. Likewise, in some examples, display devices 110-140 may additionally or alternatively be in wireless communication (e.g., directly or indirectly) with partner devices 136 and/or server system 134. Various couplings shown in FIG. 1 can be facilitated with wireless access point (WAP) 138, as also mentioned below.

In certain embodiments, analyte sensor electronics module 12 includes electronic circuitry associated with measuring and processing analyte sensor data or information, including prospective algorithms associated with processing and/or calibration of the analyte sensor data/information. Analyte sensor electronics module 12 can be physically/mechanically connected to analyte sensor 10 and can be integral with (non-releasably attached to) or releasably attachable to analyte sensor 10. Analyte sensor electronics module 12 may also be electrically coupled to analyte sensor 10, such that the components may be electromechanically coupled to one another. Analyte sensor electronics module 12 may include hardware, firmware, and/or software that enables measurement and/or estimation of levels of the analyte in a host/user via analyte sensor 10 (e.g., which may be/include a glucose sensor). For example, analyte sensor electronics module 12 can include one or more of a potentiostat, a power source for providing power to analyte sensor 10, other components useful for signal processing and data storage, and a telemetry module for transmitting data from the sensor electronics module to one or more display devices. Electronics can be affixed to a printed circuit board (PCB) within analyte sensor system 8, or platform or the like, and can take a variety of forms. For example, the electronics can take the form of an integrated circuit (IC), such as an Application-Specific Integrated Circuit (ASIC), a microcontroller, a processor, and/or a state machine.

Analyte sensor electronics module 12 may include sensor electronics that are configured to process sensor information, such as sensor data, and generate transformed sensor data and displayable sensor information. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, all of which are incorporated herein by reference in their entireties.

With further reference to FIG. 1, display devices 110, 120, 130, and/or 140 can be configured for displaying (and/or alarming) displayable sensor information that may be transmitted by analyte sensor electronics module 12 (e.g., in a customized data package that is transmitted to the display devices based on their respective preferences). Each of display devices 110, 120, 130, or 140 can (respectively) include a display such as touchscreen display 112, 122, 132,/or 142 for displaying sensor information and/or analyte data to a user and/or receiving inputs from the user. For example, a graphical user interface (GUI) may be presented to the user for such purposes. In embodiments, the display devices may include other types of user interfaces such as voice user interface instead of or in addition to a touchscreen display for communicating sensor information to the user of the display device and/or receiving user inputs. In embodiments, one, some, or all of display devices 110, 120, 130, 140 may be configured to display or otherwise communicate the sensor information as it is communicated from analyte sensor electronics module 12 (e.g., in a data package that is transmitted to respective display devices), without any additional prospective processing required for calibration and/or real-time display of the sensor data.

The plurality of display devices 110, 120, 130, 140 depicted in FIG. 1 may include a custom display device, for example, analyte display device 110, specially designed for displaying certain types of displayable sensor information associated with analyte data received from analyte sensor electronics module 12 (e.g., a numerical value and/or an arrow, in embodiments). In embodiments, one of the plurality of display devices 110, 120, 130, 140 includes a smartphone, such as a mobile phone, based on an Android, IOS, or other operating system, and configured to display a graphical representation of the continuous sensor data (e.g., including current and/or historic data).

As further illustrated in FIG. 1 and mentioned above, analyte monitoring system 100 may also include WAP 138 that may be used to couple one or more of analyte sensor system 8, the plurality display devices 110, 120, 130, 140 etc., server system 134, and partner devices 136 to one another. For example, WAP 138 may provide WiFi and/or cellular or other wireless connectivity within analyte monitoring system 100. Near Field Communication (NFC) may also be used among devices of analyte monitoring system 100 for exchanging data, as well as for performing specialized functions, e.g., waking up or powering a device or causing the device (e.g., analyte sensor electronics module 12 and/or a transmitter) to exit a lower power mode or otherwise change states and/or enter an operational mode. Server system 134 may be used to collect analyte data from analyte sensor system 8 and/or the plurality of display devices, for example, to perform analytics thereon, generate universal or individualized models for glucose levels and profiles, provide services or feedback, including from individuals or systems remotely monitoring the analyte data, and so on.

Partner device(s) 136, by way of overview and example, can usually communicate (e.g., wirelessly) with analyte sensor system 8, including for authentication of partner device(s) 136 and/or analyte sensor system 8, as well as for the exchange of analyte data, medicament data, other data, and/or control signaling or the like. Partner devices 136 may include a passive device in example embodiments of the disclosure. One example of partner device 136 may be an insulin pump for administering insulin to a user in response and/or according to an analyte level of the user as measured/approximated using analyte sensor system 8. For a variety of reasons, it may be desirable for such an insulin pump to receive and track glucose values transmitted from analyte sensor system 8 (with reference to FIG. 1 for example). One example reason for this is to provide the insulin pump a capability to suspend/activate/control insulin administration to the user based on the user's glucose value being below/above a threshold value.

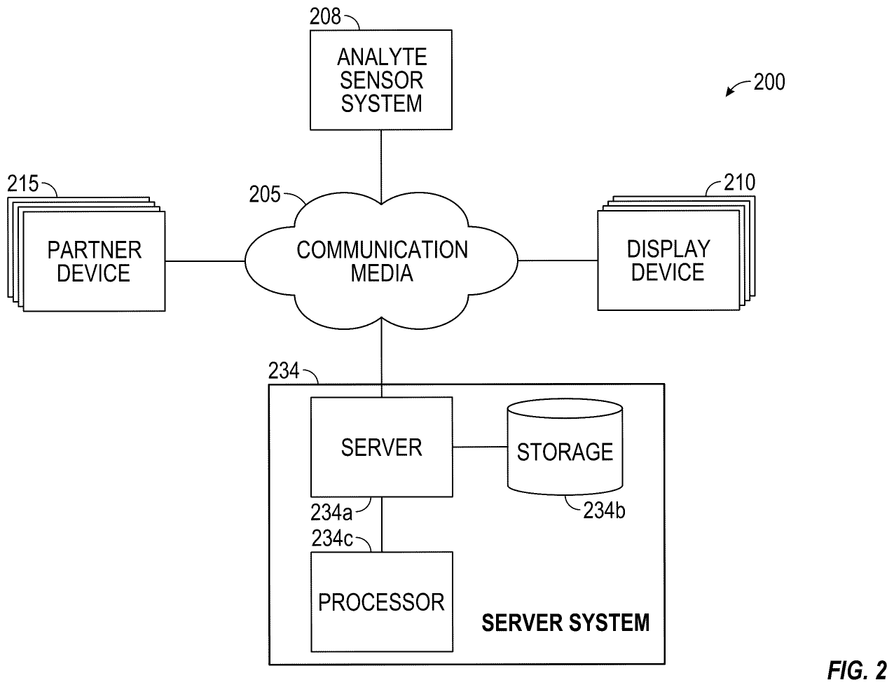

Referring now to FIG. 2, a health monitoring and management system 200 is depicted. Health monitoring and management system 200 may be used in connection with implementing embodiments of the disclosed systems, methods, apparatuses, and/or devices, including, for example, aspects described above in connection with FIG. 1. By way of example, various below-described components of FIG. 2 may be used to provide wireless communication of analyte (e.g., glucose) data, for example among/between analyte sensor system 208, display devices 210, partner devices 215, and/or one or more server systems 234, and so on. In some cases, analyte sensor system 208 illustrated in FIG. 2 may be an example of the analyte sensor system 8 illustrated in FIG. 1. Additionally, in some cases, the display devices 210 illustrated in FIG. 2 may be examples of the display devices 110, 120, 130, and 140 illustrated in FIG. 1. Additionally, in some cases, partner devices 215 illustrated in FIG. 2 may be examples of the partner device 136 illustrated in FIG. 1.

As shown in FIG. 2, health monitoring and management system 200 may include analyte sensor system 208, one or more display devices 210, and/or one or more partner devices 215. Additionally, in the illustrated embodiment, health monitoring and management system 200 includes server system 234, which can in turn include server 234a coupled to processor 234c and storage 234b. Analyte sensor system 208 may be coupled to display devices 210, partner devices 215, and/or server system 234 via communication media 205. Some details of the processing, gathering, and exchanging of data, and/or executing actions (e.g., providing medicaments or related instructions) by analyte sensor system 208, partner devices 215, and/or display device 210, etc., are provided below. Herein, display devices 210, partner devices 215, and server system 234 may be referred to as display devices and may be configured to communicate with analyte sensor system 208.

Analyte sensor system 208, display devices 210, and/or partner devices 215 may exchange messaging (e.g., control signaling) via communication media 205, and communication media 205 may also be used to deliver analyte data to display devices 210, partner devices 215, and/or server system 234. As alluded to above, display devices 210 may include a variety of electronic computing devices, such as a smartphone, tablet, laptop, wearable device, etc. Display devices 210 may also include analyte display device 110 that may be customized for the display and conveyance of analyte data and related notifications etc. Partner devices 215 may include medical devices, such as an insulin pump or pen, connectable devices, such as a smart fridge or mirror, key fob, and other devices.

In embodiments, communication media 205 may implemented using one or more wireless communication protocols, such as for example BLUETOOTH, BLUETOOTH Low Energy (BLE), ZigBee, WiFi, IEEE 802.11 protocols, Infrared (IR), Radio Frequency (RF), 2G, 3G, 4G, 5G, etc., and/or wired protocols and media. It will also be appreciated upon studying the present disclosure that communication media can be implemented as one or more communication links, including in some cases, separate links, between the components of health monitoring and management system 200, whether or not such links are explicitly shown in FIG. 2 or referred to in connection therewith. By way of illustration, analyte sensor system 208 may be coupled to display device 210 via a first link of communication media 205 using BLE, while analyte sensor system 208 may be coupled to server system 234 by a second link of communication media 205 using a WiFi communication protocol. In embodiments, a BLE signal may be temporarily attenuated to minimize data interceptions. For example, attenuation of a BLE signal through hardware or firmware design may occur temporarily during moments of data exchange (e.g., pairing).

In embodiments, the elements of health monitoring and management system 200 may be used to perform operations of various processes described herein and/or may be used to execute various operations and/or features described herein with regard to one or more disclosed systems and/or methods. Upon studying the present disclosure, one of skill in the art will appreciate that health monitoring and management system 200 may include single or multiple analyte sensor systems 208, communication media 205, and/or server systems 234.

As mentioned, communication media 205 may be used to connect or communicatively couple analyte sensor system 208, display devices 210, partner devices 215, and/or server system 234 to one another or to a network. Communication media 205 may be implemented in a variety of forms. For example, communication media 205 may include one or more of an Internet connection, such as a local area network (LAN), a person area network (PAN), a wide area network (WAN), a fiber optic network, internet over power lines, a hard-wired connection (e.g., a bus), DSL, and the like, or any other kind of network connection or communicative coupling. Communication media 205 may be implemented using any combination of routers, cables, modems, switches, fiber optics, wires, radio (e.g., microwave/RF, AM, FM links etc.), and the like. Upon reading the present disclosure, one of skill in the art will recognize other ways to implement communication media 205 for communications purposes and will also recognize that communication media 205 may be used to implement features of the present disclosure using as of yet undeveloped communications protocols that may be deployed in the future.

Further referencing FIG. 2, server 234a may receive, collect, and/or monitor information, including analyte data, medicament data, and related information, from analyte sensor system 208, partner devices 215 and/or display devices 210, such as input responsive to the analyte data or medicament data, or input received in connection with an analyte monitoring application running on analyte sensor system 208 or display device 210, or a medicament delivery application running on display device 210 or partner device 215. As such, server 234a may receive, collect, and/or monitor information from partner devices 215, such as, for example, information related to the provision of medicaments to a user and/or information regarding the operation of one or more partner devices 215. Server 234a may also receive, collect, and/or monitor information regarding a user of analyte sensor system 208, display devices 210, and/or partner devices 215.

In embodiments, server 234a may be adapted to receive such information via communication media 205. This information may be stored in storage 234b and may be processed by processor 234c. For example, processor 234c may include an analytics engine capable of performing analytics on information that server 234a has collected, received, etc. via communication media 205. In embodiments, server 234a, storage 234b, and/or processor 234c may be implemented as a distributed computing network, such as a Hadoop RTM network, or as a relational database or the like. The aforementioned information may then be processed at server 234a such that services may be provided to analyte sensor system 208, display devices 210, partner devices 215, and/or a user(s) thereof. For example, such services may include diabetes management feedback for the user.

Server 234a may include, for example, an Internet server, a router, a desktop or laptop computer, a smartphone, a tablet, a processor, a module, or the like, and may be implemented in various forms, including, for example, an integrated circuit or collection thereof, a printed circuit board or collection thereof, or in a discrete housing/package/rack or multiple of the same. In embodiments, server 234a at least partially directs communications made over communication media 205. Such communications may include the delivery of analyte data, medicament data, and/or messaging related thereto (e.g., advertisement, authentication, command, or other messaging). For example, server 234a may process and exchange messages between and/or among analyte sensor system 208, display devices 210, and/or partner devices 215 related to frequency bands, timing of transmissions, security/encryption, alarms, alerts, notifications, and so on. Server 234a may update information stored on analyte sensor system 208, partner devices 215, and/or display devices 210, for example, by delivering applications thereto or updating the same, and/or by reconfiguring system parameters or other settings of analyte sensor system 208, partner devices 215, and/or display devices 210. Server 234a may send/receive information to/from analyte sensor system 208, partner devices 215, and/or display devices 210 in real time, periodically, sporadically, or on an event-drive basis. Further, server 234a may implement cloud computing capabilities for analyte sensor system 208, partner devices 215, and/or display devices 210.

With the above description of aspects of the presently disclosed systems and methods for wireless communication of analyte data, examples of some specific features of the present disclosure will now be provided. It will be appreciated by one of skill in the art upon studying the present disclosure that these features may be implemented using aspects and/or combinations of aspects of the example configurations described above, whether or not explicit reference is made to the same.

### Analyte Data

Referring back to FIG. 1, as mentioned above, in embodiments, analyte sensor system 8 is provided for measurement of an analyte in a host or user. By way of an overview and an example, analyte sensor system 8 may be implemented as an encapsulated microcontroller that makes sensor measurements, generates analyte data (e.g., by calculating values for continuous glucose monitoring data), and engages in wireless communications (e.g., via Bluetooth and/or other wireless protocols) to send such data to remote devices (e.g., display devices 110, 120, 130, 140, partner devices 136, and/or server system 134).

Analyte sensor system 8 may include: analyte sensor 10 configured to measure a concentration or level of the analyte in the host, and analyte sensor electronics module 12 that is typically physically connected to analyte sensor 10 before analyte sensor 10 is implanted in a user. In some cases, the analyte sensor 10 may be a single-analyte sensor or a multi-analyte sensor capable of measuring one or more analytes, such as glucose, lactate, potassium, and the like. In embodiments, analyte sensor electronics module 12 includes electronics configured to process a data stream associated with an analyte concentration measured by analyte sensor 10, in order to generate sensor information that includes raw sensor data, transformed sensor data, and/or any other sensor data, for example. Analyte sensor electronics module 12 may further be configured to generate analyte sensor information that is customized for respective display devices 110, 120, 130, 140, partner devices 136, and/or server system 134. Analyte sensor electronics module 12 may further be configured such that different devices may receive different sensor information and may further be configured to wirelessly transmit sensor information to such display devices 110, 120, 130, 140, partner devices 136, and/or server system 134.

The term “analyte” as used herein is a broad term and is to be given its ordinary and customary meaning to a person of ordinary skill in the art (and is not to be limited to a special or customized meaning), and furthermore refers without limitation to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. In some embodiments, the analyte for measurement by the sensor heads, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-hydroxy-cholic acid; cortisol; creatine kinase; creatine kinase MM isoenzyme; cyclosporin A; d-penicillamine; deethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1,); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniac, Myoglobin, Onchocerca volvulus, parainfluenza virus, Plasmodium falciparum, poliovirus, Pseudomonas aeruginosa, respiratory syncytial virus, Rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferring; UDP-galactose-4-epimerase; urca; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments. The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

### Analyte Sensor System

As described to above with reference to FIG. 1, in some embodiments, analyte sensor 10 includes a continuous glucose sensor, for example, a subcutaneous, transdermal (e.g., transcutaneous), or intravascular device. In embodiments, such a sensor or device can continuously measure and analyze glucose measurements in the interstitial fluid, blood samples, etc., depending on whether the device is subcutaneous, transdermal, or intravascular. Analyte sensor 10 can use any method of analyte measurement, including for example glucose-measurement, including enzymatic, chemical, physical, electrochemical, spectrophotometric, polarimetric, calorimetric, iontophoretic, radiometric, immunochemical, and the like.

In embodiments where analyte sensor 10 is a glucose sensor, analyte sensor 10 can use any method, including invasive, minimally invasive, and non-invasive sensing techniques (e.g., fluorescence monitoring), or the like, to provide a data stream indicative of the concentration of glucose in a host. The data stream may be a raw data signal, which may be converted into a calibrated and/or filtered data stream that can be used to provide a useful value of glucose to a user, such as a patient or a caretaker (e.g., a parent, a relative, a guardian, a teacher, a doctor, a nurse, or any other individual that has an interest in the wellbeing of the host).

A glucose sensor can be any device capable of measuring the concentration of glucose. According to one example embodiment described below, an implantable glucose sensor may be used. However, it should be understood that the devices and methods described herein can be applied to any device capable of detecting a concentration of an analyte, glucose for example, and providing an output signal that represents the concentration of the analyte, again glucose for example (e.g., as a form of analyte data).

In embodiments, analyte sensor 10 is an implantable glucose sensor, such as described with reference to U.S. Pat. No. 6,001,067 and U.S. Patent Publication No. US-2005-0027463-A1. In embodiments, analyte sensor 10 is a transcutaneous glucose sensor, such as described with reference to U.S. Patent Publication No. US-2006-0020187-A1. In embodiments, analyte sensor 10 is configured to be implanted in a host vessel or extracorporeally, such as is described in U.S. Patent Publication No. US-2007-0027385-A1, co-pending U.S. Patent Publication No. US-2008-0119703-A1 filed Oct. 4, 2006, U.S. Patent Publication No. US-2008-0108942-A1 filed on Mar. 26, 2007, and U.S. Patent Application No. US-2007-0197890-Al filed on Feb. 14, 2007. In embodiments, the continuous glucose sensor includes a transcutaneous sensor such as described in U.S. Pat. No. 6,565,509 to Say et al., for example. In embodiments, analyte sensor 10 is a continuous glucose sensor that includes a subcutaneous sensor such as described with reference to U.S. Pat. No. 6,579,690 to Bonnecaze et al. or U.S. Pat. No. 6,484,046 to Say et al., for example. In embodiments, the continuous glucose sensor includes a refillable subcutaneous sensor such as described with reference to U.S. Pat. No. 6,512,939 to Colvin et al., for example. The continuous glucose sensor may include an intravascular sensor such as described with reference to U.S. Pat. No. 6,477,395 to Schulman et al., for example. The continuous glucose sensor may include an intravascular sensor such as described with reference to U.S. Pat. No. 6,424,847 to Mastrototaro et al., for example.

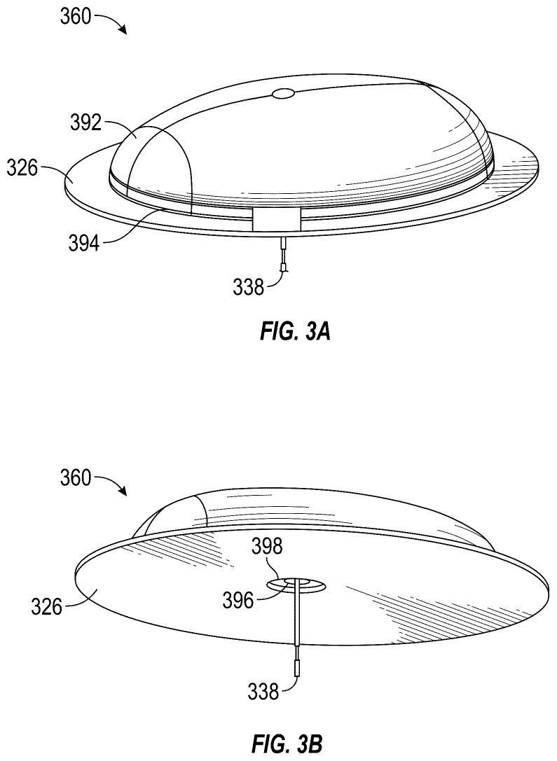

FIG. 3A illustrates a perspective view of an on-skin sensor assembly 360 that may be used in connection with the analyte sensor system 8 of FIG. 1 and/or the analyte sensor system 208 of FIG. 2. For example, on-skin sensor assembly 360 may be or include analyte sensor system 8 and/or analyte sensor system 208. On-skin sensor assembly 360 may include an outer housing with a first, top portion 392 and a second, bottom portion 394. In embodiments, the outer housing may include a clamshell design. On-skin sensor assembly 360 may include, for example, similar components as analyte sensor electronics module 12 described above in connection with FIG. 1, for example, a potentiostat, a power source for providing power to analyte sensor 10, signal processing components, data storage components, and a communication module (e.g., a telemetry module) for one-way or two-way data communication, a printed circuit board (PCB), an integrated circuit (IC), an Application-Specific Integrated Circuit (ASIC), a microcontroller, and/or a processor.

As shown in FIG. 3A, the outer housing may feature a generally oblong shape. The outer housing may further include aperture 396 disposed substantially through a center portion of outer housing and adapted for sensor 338 and needle insertion through a bottom of on-skin sensor assembly 360. In embodiments, aperture 396 may be a channel or elongated slot. On-skin sensor assembly 360 may further include an adhesive patch 326 configured to secure on-skin sensor assembly 360 to skin of the host. In embodiments, adhesive patch 326 may include an adhesive suitable for skin adhesion, for example a pressure sensitive adhesive (e.g., acrylic, rubber-based, or other suitable type) bonded to a carrier substrate (e.g., spun lace polyester, polyurethane film, or other suitable type) for skin attachment, though any suitable type of adhesive is also contemplated. As shown, adhesive patch 326 may feature an aperture 398 aligned with aperture 396 such that sensor 338 may pass through a bottom of on-skin sensor assembly 360 and through adhesive patch 326.

FIG. 3B illustrates a bottom perspective view of on-skin sensor assembly 360 of FIG. 3A. FIG. 3B further illustrates aperture 396 disposed substantially in a center portion of a bottom of on-skin sensor assembly 360, and aperture 398, both adapted for sensor 338 and needle insertion.

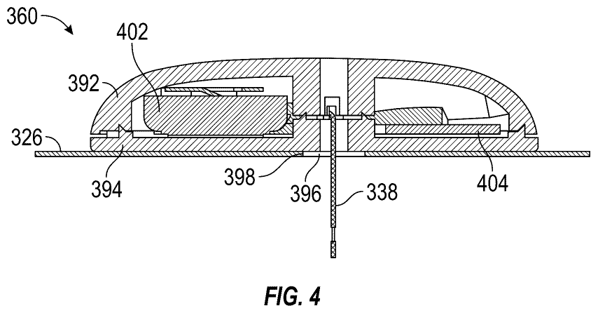

FIG. 4 illustrates a cross-sectional view of on-skin sensor assembly 360 of FIGS. 3A and 3B. FIG. 4 illustrates first, top portion 392 and second, bottom portion 394 of the outer housing, adhesive patch 326, aperture 396 in the center portion of on-skin sensor assembly 360, aperture 398 in the center portion of adhesive patch 326, and sensor 338 passing through aperture 396. The electronics unit, previously described in connection with FIG. 3A, may further include circuit board 404 and battery 402 configured to provide power to at least circuit board 404.

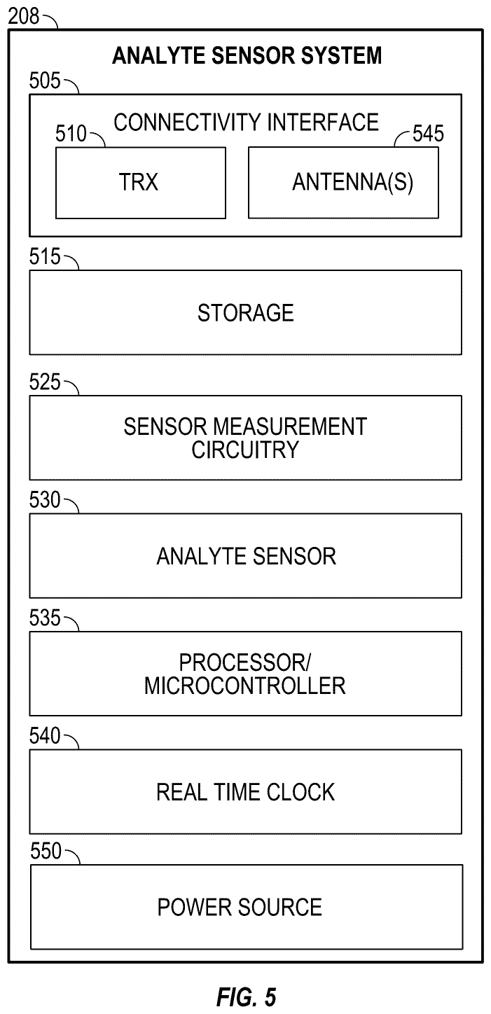

Turning now to FIG. 5, a more detailed functional block diagram of analyte sensor system 208 (discussed above, for example, in connection with FIGS. 1 and 2) is provided. As noted above, the analyte sensor system 208 may be an example of the analyte sensor system 8 illustrated in FIG. 1. As shown in FIG. 5, analyte sensor system 208 may include an analyte sensor 530 (e.g., which may be an example of the analyte sensor 10 illustrated in FIG. 1) coupled to sensor measurement circuitry 525 for receiving, processing, and managing analyte data, obtained from the analyte sensor 530, indicative of analyte levels of a user of the analyte sensor system 208. Sensor measurement circuitry 525 may be coupled to processor/microcontroller 535. In some embodiments, processor/microcontroller 535 may include one or more processors and may be part of analyte sensor electronics module 12 in FIG. 1. In some embodiments, processor/microcontroller 535 may perform part or all of the functions of sensor measurement circuitry 525 for obtaining and processing analyte data (e.g., sensor measurement values) from the analyte sensor 530 and generating analyte data representative of the sensor measurement values. In some embodiments, the processed analyte data may be stored in storage 515, including one or more memories

Processor/microcontroller 535 may be further coupled to a radio unit or transceiver 510 (e.g., which may be part of analyte sensor electronics module 12 in FIG. 1). In some embodiments, the processor/microcontroller 535 may be configured to provide sending sensor data, such as the analyte data, and other data to the transceiver 510 for transmission to an external device, such as display device 210 (referencing FIG. 2 by way of example). The transceiver 510 may also be configured to receive, from the external device, control information including requests for certain information and commands to perform certain actions. In some cases, the transceiver 510 may include logic or circuitry for communicating (e.g., transmitting and receiving) using different communication protocols, such as BLUETOOTH, BLUETOOTH Low Energy (BLE), near-field communication (NFC), WiFi, Third Generation Partnership Project (3GPP)-based wireless communication protocols, and other wireless communication protocols. In some embodiments, the transceiver 510 may be coupled to an antenna system 545 associated with the connectivity interface 505, allowing the analyte sensor system 208 to wirelessly transmit and receive data. For example, the transceiver 510 may be configured to output data, such as the analyte data for wireless transmission via at least one main antenna of the antenna system 545 or may be configured to obtain data that is wirelessly received via at least one of the antennas of the antenna system 545. In some cases, the antenna system 545 may be tuned to a particular frequency depending on a communication protocol used for communicating data. For example, in some embodiments, the antenna system 545 may include one or more antennas tuned for communicating data via a BLE protocol (e.g., tuned to 2.4 gigahertz). In some embodiments, the antenna system 545 may include one or more antennas tuned for communicating data via an NFC protocol (e.g., tuned to 13.56 megahertz).

Analyte sensor system 208, in example implementations, gathers analyte data using the analyte sensor 530 and transmits the same or a derivative thereof to display device 310, partner device 315, and/or server system 334 using the transceiver 510 and antenna system 545. Data points regarding analyte values may be gathered and transmitted over the life of the analyte sensor 530. New measurements and/or related information may be transmitted often enough for a remote device/individual to adequately monitor analyte (e.g., glucose) levels.

It is to be appreciated that some details of the processing, gathering, and exchanging data by analyte sensor system 208, partner devices 315, and/or display device 310 etc. are provided elsewhere herein. It will be appreciated upon studying the present disclosure that analyte sensor system 208 may contain several like components that are described with respect to FIG. 1 or 2, at least for some embodiments herein. The details and uses of such like components may therefore be understood vis-a-vis analyte sensor system 208 even if not expressly described here with reference to FIG. 5.

### Aspects Related an Analyte Sensor System with Active and Passive Antennas

Patients with diabetes may benefit from real-time diabetes management guidance that is determined based on a physiological state of the patient. In certain cases, the physiological state of the patient may be determined using diagnostics systems, such as an analyte sensor system (e.g., analyte sensor system 8 and/or analyte sensor system 208). In some embodiments, analyte sensor system 208 may be configured to measure analyte levels of a patient and inform the patient about the identification and/or prediction of adverse glycemic events, such as hyperglycemia and hypoglycemia. Additionally, the analyte sensor system 208 may be configured to help inform the type of guidance provided to the patient in response to these adverse glycemic events.

For example, the analyte sensor system 208 of FIG. 5 may be worn by a patient and configured to continuously measure the analyte levels of the patient over time using a continuous analyte sensor, such as the analyte sensor 530. The measured analyte levels may then be processed by the analyte sensor system 208 (e.g., by the processor/microcontroller 535) to identify and/or predict adverse events, and/or to provide guidance to the patient for treatment and or actions to abate or prevent the occurrence of such adverse events. Analyte data indicating the analyte levels of the patient may then be output by the processor/microcontroller 535 to the transceiver 510 of the analyte sensor system 208 for wireless transmission to a communications device, such as one or more of display devices 110, 120, 130, 140 depicted and described with respect to FIG. 1 and/or the display device 210 depicted and described with respect to FIG. 2. In some embodiments, the analyte data may be transmitted to the communications device using the at least one main antenna of the antenna system 545 and a particular wireless communication protocol, such as BLUETOOTH, BLE, NFC, WiFi, 3GPP-based wireless communication protocols, or other wireless communication protocols.

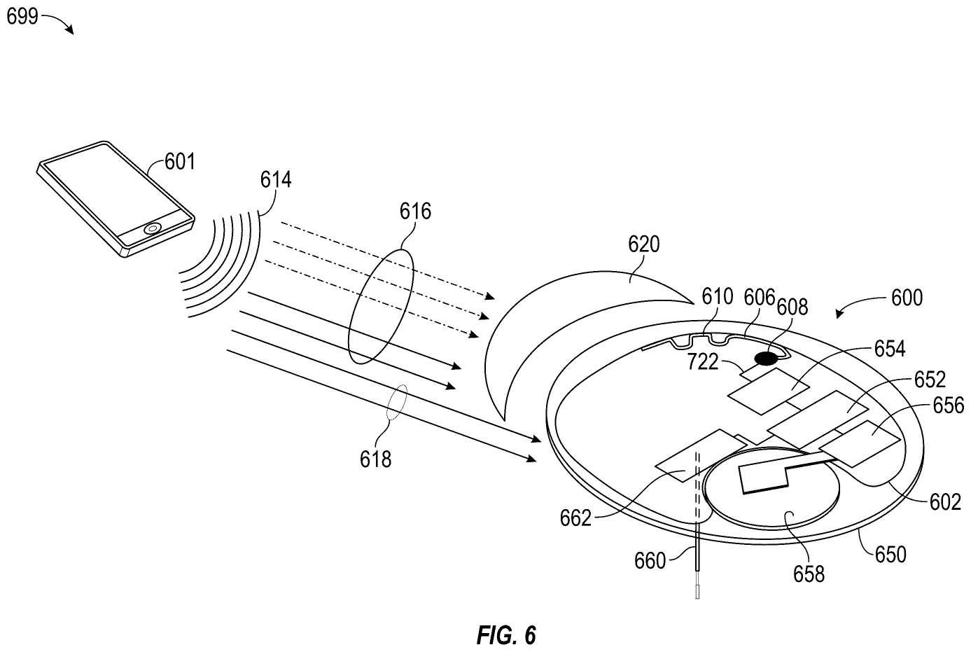

FIG. 6 illustrates an example analyte sensor system 600 having a main antenna 606 that may be used to communicate with a display device 601 in an analyte monitoring system 699. The analyte sensor system 600 may be an example of the analyte sensor system 8 depicted and described with respect to FIG. 1 and/or the analyte sensor system 208 depicted and described with respect to FIGS. 2 and 5. In certain embodiments, the analyte sensor system 600 is a continuous glucose monitoring (CGM) system. As shown, the analyte sensor system 600 includes a housing 650 that may be adhered to a body 620 of a user or patient using, for example, an adhesive patch. The housing 650 may house one or more electrical components of the analyte sensor system 600, including a printed circuit board (PCB) 602, a processor/microcontroller 652 (e.g., including one or more processors), a transceiver circuit 654, the main antenna 606, a main antenna feed 608, a storage 656 (e.g., including one or more memories), a battery 658, an analyte sensor 660, and sensor measurement circuitry 662. In some embodiments, the battery 658 may be configured to power the one or more electrical components of the analyte sensor system 600. Further, as shown in FIG. 6, the main antenna 606 may be communicatively coupled to the main antenna feed 608 and other electrical components of the analyte sensor system 600 (e.g., transceiver circuit 654, processor/microcontroller 652, analyte sensor 660, etc.). In certain embodiments, the main antenna 606 and/or the main antenna feed 608 may be part of, or representative of, the antenna system 545 of FIG. 5.

In some embodiments, the transceiver circuit 654 may be operable to send and/or receive information pertaining to the analyte sensor system 600 (e.g., send and/or receive operational information) to and/or from display device 601. In certain embodiments, the analyte sensor system is operable to send analyte data to the display device 601, indicating analyte levels associated with a user of the analyte sensor system 600. Additionally, in certain embodiments, the main antenna 606 transmits and/or receives pairing information from the display device 601. For example, the analyte sensor system 600 may receive initial pairing instructions, keep alive instructions, or disconnect instructions from the display device 601. In certain embodiments, the main antenna 606 may be designed in order to facilitate in optimal reception of radio signals from the display device 601. In some embodiments, the main antenna 606 may be integrated on a PCB.

In some embodiments, the main antenna 606 may take any form that may fit within the overall form factor of the analyte sensor system 600. FIG. 6 illustrates an embodiment where the main antenna 606 is configured as meander F-line antenna. Meander portion 610 of the main antenna 606 may be configured so as to help to miniaturize an overall length of the main antenna 606 without impacting performance of the main antenna 606. FIG. 6 illustrates two terminals of the F-section of the main antenna 606. While FIG. 6 illustrates the main antenna 606 as having a meandering F-line (e.g., meandering portion 610), other antenna types are readily envisioned (e.g., straight, curved, bent, etc.). In certain embodiments, the main antenna 606 may include, without limitation, an inverted-F antenna, a dipole antenna, a loop antenna, a monopole antenna, a fractal antenna, and combinations of the same and like.

In certain embodiments, the main antenna 606 may be operable to send and/or receive information over various radio frequencies, such as, but not limited to, BLUETOOTH, BLE, Wi-Fi, and the like. For example, as shown, the analyte sensor system 600 may be configured to receive a BLE signal 614 from display device 601. However, in some cases, due to a manner in which the analyte sensor system 600 is positioned on a body 620 of a user of the analyte sensor system 600 and/or positions of the body 620 itself, the body 620 may block the BLE signal 614 from being received by the main antenna 606. For example, as shown, the BLE signal 614 may have different signal paths, such as a first set of signal paths 616 and a second set of signal paths 618. Further, as shown, due to the positioning of the body 620, the body 620 may block the first set of signal paths 616 of the BLE signal 614, preventing the BLE signal 614 from being received by the main antenna 606. Additionally, as shown, while the second set of signal paths 618 of the BLE signal 614 may bypass the body 620, the second set of signal paths 618 of the BLE signal 614 may still fail to reach portions of the main antenna 606. Furthermore, the body 620 may also block, shadow, and/or hinder the main antenna 606 from transmitting signals to the display device 601.

As can be seen, a single antenna design for the analyte sensor systems 600 may be prone to scenarios in which signals transmitted and/or received by the analyte sensor system 600 may be blocked, shadowed, or otherwise impeded by the body 620 of the user, or other obstructions. This blocking and/or shadowing of signals (e.g., BLE signal 614) may lead to certain negative effects, such as dropped data packets, poor signal reception, and the like. Dropped packets and poor reception may lead to retransmissions of data packets, increasing power consumption at the analyte sensor system 600, which may be problematic if the analyte sensor system 600 is power-limited.

One manner to help avoid issues of blocked signals may be to design the analyte sensor system 600 with multiple active main antennas (e.g., two or more antennas that use active circuit components, such as, amplifiers, and/or are coupled to a transceiver). However, having multiple active main antennas may increase a complexity and cost associated with manufacturing the analyte sensor system 600, as additional complex components are needed to allow for switching between active main antennas. In addition, firmware for controlling the additional antennas may be needed in order to switch between multiple antennas of the analyte sensor system 600, potentially increasing power consumption and adding more cost and time to the manufacturing process.

Accordingly, aspects of the present disclosure provide techniques for avoiding and/or reducing the issues described above associated with the shadowing and/or blocking of signals transmitted between an analyte sensor system and a display device. For example, in some embodiments, in addition to using a main antenna to communicate signals between the analyte sensor system and display device, the techniques presented herein may involve equipping the analyte sensor system with a passive antenna or a relay antenna. In some embodiments, the passive antenna may be configured to passively receive signals from the display device and reflect or re-radiate these signals towards the main antenna of the analyte sensor system, allowing the main antenna of the analyte sensor system to still receive signals from the display device even though signal paths associated with the signals from the display device may be blocked to the main antenna by a body of a user of the analyte sensor system or some other obstruction. For example, with reference to FIG. 6, in some embodiments, even though the first set of signal paths 616 may be blocked by the body 620, preventing the main antenna 606 from receiving the BLE signal 614, a passive antenna may be used to receive the BLE signal 614 along the second set of signal paths 618 and reflect or re-radiate the BLE signal 614 to the main antenna 606.

Further, in some embodiments, the passive antenna may not be connected to a transceiver of the analyte sensor system and may not require additional active components to receive and re-radiate the signals from the display device unlike active antennas such as the unlike the main antenna 606 of the analyte sensor system 600 (e.g., additional circuitry components, firmware, etc.). As a result, a cost, time, and/or complexity associated with manufacturing the analyte sensor system including the passive and main antenna may be reduced relative to an analyte sensor system with multiple active antennas.

In certain embodiments, a resistive (e.g., having a resistor) and/or reactive (e.g., having an inductor and/or capacitor) loading may also be used to terminate the passive antenna and boost an ability of the passive antenna to receive and reflect the passively received signals to the main antenna that is coupled to the transceiver. For example, in certain embodiments, use of the passive antenna may increase signal strength or gain associated with the main antenna by more than 3 dB over various radio frequencies (e.g., 2.4 GHz associated with BLUETOOTH communication). This increase in signal strength is indicative of improvements in connectivity between the analyte sensor system and the display device during obstructed and/or partially obstructed use. These improvements decrease an amount of signal loss (e.g., BLE signal loss) between the analyte sensor system and the display device. This in turn results in fewer retransmissions of data between the analyte sensor system and the display device, which is otherwise battery-intensive. As a result, analyte sensor systems of the disclosure provide improved wireless connectivity and battery efficiency by conserving power required to maintain a connection with the display device and/or send/receive data to the display device.

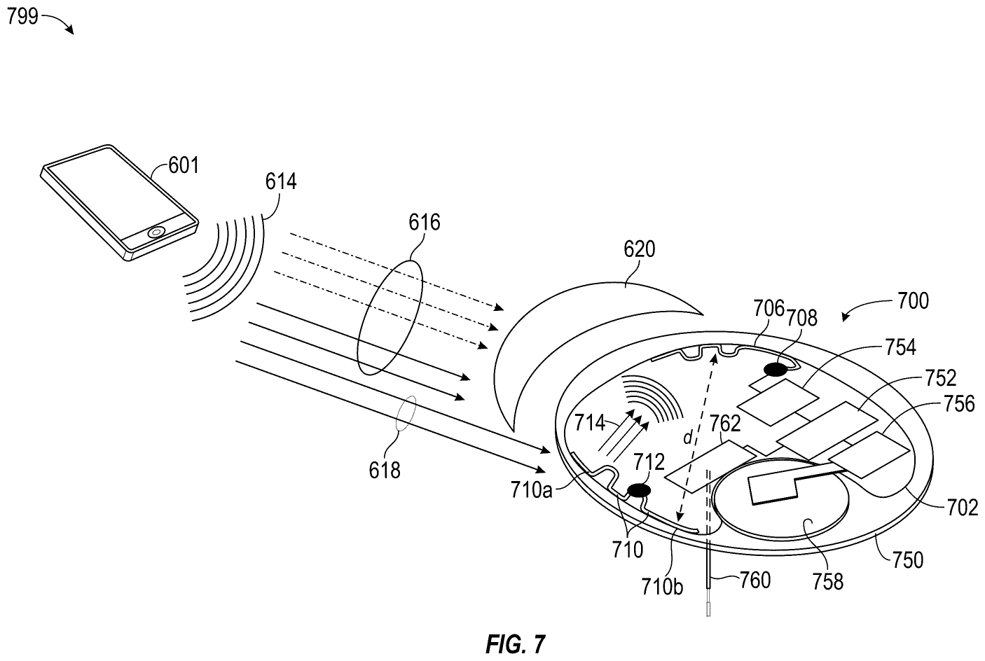

FIG. 7 illustrates an example analyte sensor system 700 having a main antenna and passive antenna for communicating with display device 601 in an analyte monitoring system 799. In certain embodiments, the display device 601 may include any of the one or more of display devices 110, 120, 130, and 140 of FIG. 1. The analyte sensor system 700 may be an example of the analyte sensor system 8 depicted and described with respect to FIG. 1 and/or the analyte sensor system 208 of FIGS. 2 and 5. In certain embodiments, the analyte sensor system 700 is a continuous glucose monitoring system.

As shown, the analyte sensor system 700 includes a housing 750 that may be adhered to the body 620 of a user using, for example, an adhesive patch. The housing 750 may house one or more electrical components of the analyte sensor system 700 for obtaining, processing, and transmitting analyte data to the display device 601 and for receiving signals from the display device 601. For example, in some embodiments, the one or more electrical components may include a PCB 702, a processor/microcontroller 752 (e.g., including one or more processors), a transceiver circuit 754, a storage 756 (e.g., including one or more memories), a battery 758, an analyte sensor 760, and sensor measurement circuitry 762. In some embodiments, the battery 758 may be configured to power the one or more electrical components of the analyte sensor system 700. In some embodiments, the processor/microcontroller 752 may be an example of the processor/microcontroller 535 illustrated and described with respect to FIG. 5. In some embodiments, the transceiver circuit 754 may be an example of the transceiver 510 illustrated and described with respect to FIG. 5. In some embodiments, the storage 756 may be an example of the storage 515 illustrated and described with respect to FIG. 5. In some embodiments, the battery 758 may be an example of the power source 550 illustrated and described with respect to FIG. 5. In some embodiments, the analyte sensor 760 may be an example of the analyte sensor 530 illustrated and described with respect to FIG. 5. In some embodiments, the sensor measurement circuitry 762 may be an example of the sensor measurement circuitry 525 illustrated and described with respect to FIG. 5.

Further, as shown, the analyte sensor system 700 includes an antenna system that includes at least a first antenna 706 and a second antenna 710. In some embodiments, the first antenna 706 may be a main antenna or active antenna, and may be communicatively coupled to a main antenna terminal 708, the transceiver circuit 754, and other electrical components of the analyte sensor system 700 (e.g., processor/microcontroller 752, storage 756, battery 758, analyte sensor 760, sensor measurement circuitry 762, etc.)) Additionally, in some embodiments, the second antenna 710 may be a passive antenna and may not be communicatively coupled to the transceiver circuit 754 or any other operational or active components, such as the processor/microcontroller 752, the storage 756, a power amplifier, or the like. In some embodiments, the first antenna 706 may be at least one of a dipole antenna, monopole antenna, a loop antenna, an inverted-F antenna, or a fractal antenna. In some embodiments, the second antenna 710 may be at least one of a dipole antenna, monopole antenna, a loop antenna, an inverted-F antenna, or a fractal antenna.

Further as shown, the second antenna 710 may include one or more antenna arms, such as a first antenna arm 710a and a second antenna arm 710b, forming the second antenna 710. In some embodiments, the first antenna arm 710a and the second antenna arm 710b of the second antenna 710 may be coupled to a passive antenna terminal 712. While FIG. 7 illustrates that the plurality of antenna arms may include two antenna arms (e.g., the first antenna arm 710a and the second antenna arm 710b), it should be understood that the plurality of antenna arms may include any number of antenna arms, such as one antenna arm, three antenna arms, or more antenna arms. In certain embodiments, the first antenna 706 may be substantially similar to the main antenna 606 as described in FIG. 6.

In certain embodiments, the analyte sensor 760 of the analyte sensor system 700 may be configured to measure analyte levels of a user of the analyte sensor system 700 and provide these measurements to the processor/microcontroller 752 and/or the storage 756 of the analyte sensor system 700. For example, in some embodiments, the processor/microcontroller 752 may be configured to receive and process the measurements from the analyte sensor 760 (e.g., via sensor measurement circuitry 762). In certain embodiments, the processor/microcontroller 752 may be further configured to generate analyte data (e.g., estimated analyte values) based on the measurements received from the analyte sensor 760 and transmit, via the transceiver circuit 754, the analyte data to a display device 601.

In some embodiments, the PCB 702 may be configured to operatively couple the one or more electronic components of the analyte sensor system 700 with the first antenna 706. For example, in some embodiments, the PCB 702 may be configured to operatively couple the analyte sensor 760, the sensor measurement circuitry 762, the processor/microcontroller 752, the storage 756, the battery 658, the transceiver circuit 754, and the first antenna 706. In some embodiments, the first antenna 706 may be configured to receive signals from the display device 601 that may include, without limitation, operational instructions, configuration instructions, and combinations of the same and like. In some embodiments, the first antenna 706 may be configured to transmit, to the display device 601, a first signal including at least the analyte data, and receive, from the display device 601, a second signal (e.g., BLE signal 614) including operational instructions. Further, in some embodiments, the second antenna 710 may be configured to receive the first signal from the first antenna 706 and re-radiate the first signal towards the display device 601. In some embodiments, the second antenna 710 may also be configured to receive the second signal from the display device 601 and re-radiate the second signal towards the first antenna 706. In certain embodiments, the PCB 702 may operatively connect the analyte sensor 760 with the first antenna 706. In some embodiments, the operational instructions include at least one of configuration instructions, initial pairing instructions, keep alive instructions, disconnect instructions, or instructions to transmit the analyte data.

As noted above, in some embodiments, the one or more electronic components of the analyte sensor system 700 includes the transceiver circuit 754, which may be coupled to the first antenna 706. In some embodiments, the transceiver circuit 754 may be configured to transmit the first signal via the first antenna 706. The transceiver circuit 754 may also be configured to receive, via the first antenna 706, at least one of the second signal from the display device 601 or the re-radiated signal from the second antenna 710. In some embodiments, the transceiver circuit 754 may be configured to communicate signals, including the first signal, the second signal, and the re-radiated second signal, according to a wireless communication technology. In some embodiments, the wireless communication technology includes at least one of BLUETOOTH Low Energy (BLE), BLUETOOTH, or Wi-Fi.

As illustrated in FIG. 7, the second antenna 710 may be included on the PCB 702. However, in other embodiments, the second antenna 710 may include portions extending off the PCB 702. For example, and not by way of limitation, the second antenna 710, or portions thereof, may partially extend to an outside portion of the housing 750 of the analyte sensor system 700 (e.g., the outside housing described with respect to FIG. 3A). In certain embodiments, the second antenna 710 may not be included on the PCB 702. For example, in some embodiments, the second antenna 710 may be included on an interior or exterior surface of the housing 750 of the analyte sensor system 700. In some embodiments, the first antenna 706 and the second antenna 710 may be included on a top surface or a bottom surface of the PCB 702. In some embodiments, the second antenna 710 may be included on the bottom surface of the PCB 702 and the first antenna 706 is included on the top surface of the PCB 702, or vice versa.

In certain embodiments, the first antenna 706 and the PCB 702 may be included within the housing 750 of the analyte sensor system 700. In some embodiments, the second antenna 710 may be included in the housing 750 of the analyte sensor system 700. In some embodiments, the second antenna 710 may be disposed outside of the housing 750 of the analyte sensor system 700. For example, in some embodiments, the second antenna 710 may be incorporated into an adhesive patch attached to an outside of the housing 750 of the analyte sensor system 700.

In certain embodiments, the passive antenna terminal 712 may include one or more passive components. In certain embodiments, the one or more passive components may include, without limitation, one or more resistors, one or more capacitors, one or more inductors, or a combination thereof. In some embodiments, the second antenna 710 may be grounded at the passive antenna terminal 712. In some embodiments, an electrical property of the one or more passive electrical components may be based on at least one of a topology of the passive antenna or a distance from the passive antenna to the main antenna. In some embodiments, the electrical property may include, for example, at least one of a resistance value for one or more resistors in the one or more passive components, a capacitance value for one or more capacitors in the one or more passive components, or an inductance value for one or more capacitors in the one or more passive components.

As noted above, the second antenna 710 may be configured to receive the second signal (e.g., BLE signal 614) from the display device 601 that would otherwise be blocked and/or shadowed from the first antenna 706 by the body 620 and reflect or re-radiate the radio signal towards the first antenna 706. For example, FIG. 7 illustrates the BLE signal 614 that may be transmitted by the display device 601 along different signal paths, such as first set of signal paths 616 and second set of signal paths 618. As shown, the body 620 blocks the first set of signal paths 616 of the BLE signal 614 from the first antenna 706, similar to that as described with respect to FIG. 6. However, as shown, the second set of signal paths 618 of the BLE signal 614 may bypass the body 620, allowing the BLE signal 614 to be received by the second antenna 710. The BLE signal 614 received by the second antenna 710 may then reflected or re-radiated by the second antenna 710 towards the first antenna 706 in the form of re-radiated BLE signal 714, allowing the first antenna 706 to (indirectly) receive the BLE signal 614 from the display device 601.

In some embodiments, the ability of the second antenna 710 to reflect or re-radiate radio signals towards the first antenna 706 or the display device 601 may be based on, for example, a design of the second antenna 710 and/or the one or more passive components included within the passive antenna terminal 712 (e.g., resistors, inductors, capacitors, etc.).

In some embodiments, the second antenna 710 may be configured to match a corresponding geometry of the first antenna 706 such that signals transmitted to and/or received from the first antenna 706 of the analyte sensor system 700 provide expanded coverage to include signal paths that are blocked by the body 620. In some embodiments, the configuration may include, for example, a topology that improves or maximizes a radiated coupling and/or communication between the first antenna 706 and the second antenna 710. In some embodiments, the configuration may include, without limitation, one or more antenna arms, shapes corresponding to the first antenna 706, curved and/or straight architectures around open areas of the PCB 702, or combinations thereof. In some embodiments, a geometry of the second antenna 710 may be configured based on a topology to optimize communications between the main antenna and the display device 601. In some embodiments, a geometry of the first antenna 706 may be configured to maximize reception of the second signal from the display device 601.

As noted above, in some embodiments, the second antenna 710 includes one or more antenna arms, as described above (e.g., first antenna arm 710a and second antenna arm 710b). In certain embodiments, each antenna arm of the plurality of antenna arms of the second antenna 710 may terminate into the passive antenna terminal 712. In certain embodiments, each of the one or more antenna arms may be geometrically configured to provide optimal radiated coupling and/or communication between the first antenna 706 and the second antenna 710. In general, the second antenna 710 may take any form and/or shape to fit within the overall form factor of the analyte sensor system 700.

In some embodiments, the first antenna arm 710a and the second antenna arm 710b may be similar or different in length and/or shape. Additionally, the first antenna arm 710a and the second antenna arm 710b may also be straight, curved, and/or bent. In certain embodiments, the first antenna arm 710a and the second antenna arm 710b may be configured such that a radiation pattern is directed towards the first antenna 706, and to be able to receive radio signals, such as the BLE signal 614, from the display device 110. In certain embodiments, the first antenna arm 710a and the second antenna arm 710b may be in the form of meander lines and/or fractals to reduce length without impacting performance.

In certain embodiments, the second antenna 710 may be positioned in a particular manner so as to optimize reception of signals, such as the BLE signal 614, on alternative signal paths (e.g., the second set of signal paths 618) when signal paths associated with the first antenna 706 (e.g., the first set of signal paths 616) are blocked. In some embodiments, the second antenna 710 may be positioned in a particular manner so as to enhance reception (or transmission) performance associated with the first antenna 706 even when the signal paths associated with the first antenna 706 are not blocked. For example, in certain embodiments, the second antenna 710 may be located/positioned at a diagonal distance, d (shown in FIG. 7), from the first antenna 706 to provide more location-diversity and to minimize the probability that the first antenna 706 and the second antenna 710 are blocked at the same time. As such, in some embodiments, a geometry of the second antenna 710 may be configured to maximize a diagonal distance, d, from the first antenna 706 to a portion of the second antenna 710.

As noted above, the second antenna 710 may be coupled to the passive antenna terminal 712. Additionally, as noted above, the passive antenna terminal 712 may include one or more passive components, such as one or more resistors, one or more capacitors, and/or one or more inductors. In some embodiments, resistance values, capacitance values, and/or inductance values of the one or more passive components of the passive antenna terminal 712 may depend on a topology of the second antenna 710, as well the distance, d, from the first antenna 706. Such configurations may maximize the re-radiated BLE signal 714 by eliminating or reducing impedance mismatch between the second antenna 710 and the passive antenna terminal 712.

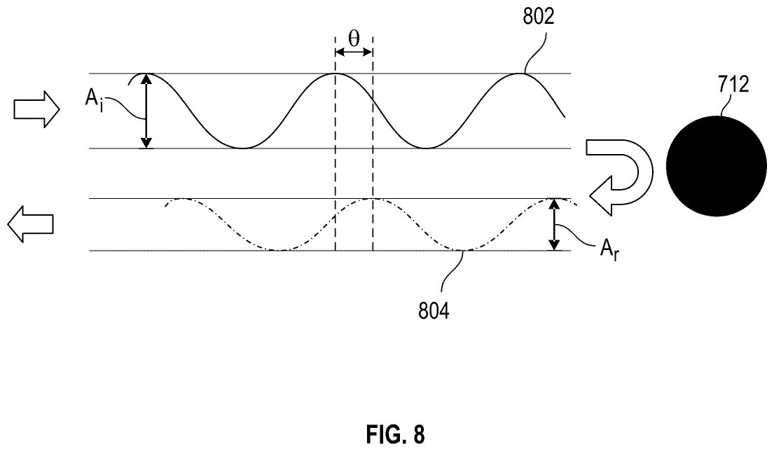

In some cases, as illustrated in FIG. 8, one or more properties of the BLE signal 614 (e.g., the second signal described above) received from the display device 601 may be adjusted as a result of being re-radiated by the second antenna 710 towards the first antenna 706. For example, FIG. 8 illustrates an example incident wave 802 and a re-radiated wave 804 at the passive antenna terminal 712 of FIG. 7. FIG. 8 is described with respect to FIG. 7 for clarity. In certain embodiments, the incident wave 802 may comprise the second signal (e.g., the BLE signal 614) received from the display device 601 illustrated and described with respect to FIG. 7. As shown, the incident wave 802 has an amplitude Aᵢ. Further, as shown, when the incident wave 802 of the BLE signal 614 is re-radiated as the re-radiated wave 804 by the second antenna 710, the re-radiated wave 804 may be re-radiated with an amplitude Aᵣ. In some embodiments, the amplitudes Aᵢ and Aᵣ may be the same. In some embodiments, the amplitudes Aᵢ and Aᵣ may be different. For example, in some embodiments, the amplitude Aᵣ may be lower than the amplitude Aᵢ. In some embodiments, the second antenna 710 may be configured to receive the incident wave 802 and re-reradiate the wave towards the first antenna 706 with a same wavelength. In some embodiments, the second antenna 710 may be configured to receive the incident wave 802 and re-reradiate the wave towards the first antenna 706 with a different wavelength.

Additionally, as shown, re-radiation of the incident wave 802 by the second antenna 710 may result in a phase shift between the incident wave 802 and the re-radiated wave 804. For example, the incident wave 802 may be received by the second antenna 710 with a first phase θᵢ while the re-radiated wave 804 may be re-radiated by the second antenna 710 with a second phase θᵣ, where the phase shift is equal to the difference between θᵢ and θᵣ. In some embodiments, the second antenna 710 may be configured to receive incident wave 802 and re-reradiate the wave towards the first antenna 706 with a same phase θ. In some embodiments, the incident wave 802 and the re-radiated wave 804 may have the same magnitude. In certain embodiments, the second antenna 710 and/or the passive antenna terminal 712 may be configured to modify the magnitude of the re-radiated wave 804.

In some embodiments, the incident wave 802 may be a wave from a portion of the BLE signal 614 (e.g., the second set of signal paths 618) that reaches the second antenna 710. When the incident wave 802 reaches the second antenna 710 and/or passive antenna terminal 712, the incident wave 802 is re-radiated as re-radiated wave 804 towards the first antenna 706 and/or the main antenna terminal 708. In certain embodiments, the incident wave 802 may be a wave from BLE signal 614 received via the second set of signal paths 618, and the re-radiated wave 804 may be a wave from the re-radiated BLE signal 714.

While FIGS. 7 and 8 illustrate the BLE signal 614 being received by the analyte sensor system 700, in certain embodiments, the operating principals of the second antenna 710 work bi-directionally. For example, a radio transmitter (e.g., BLE transmitter) may propagate a radio signal from the radio transmitter such that the propagated wave leaving the radio transmitter functions in a similar manner as the incident wave 802. In such embodiments, the second antenna 710 may reflect and re-radiate the radio signal to the display device 601 (i.e., opposite wave direction as described with respect to receiving radio signals). As such, the second antenna 710 may provide continuous and consistent communication between the analyte sensor system 700 and the display device 601.

### Example Operations

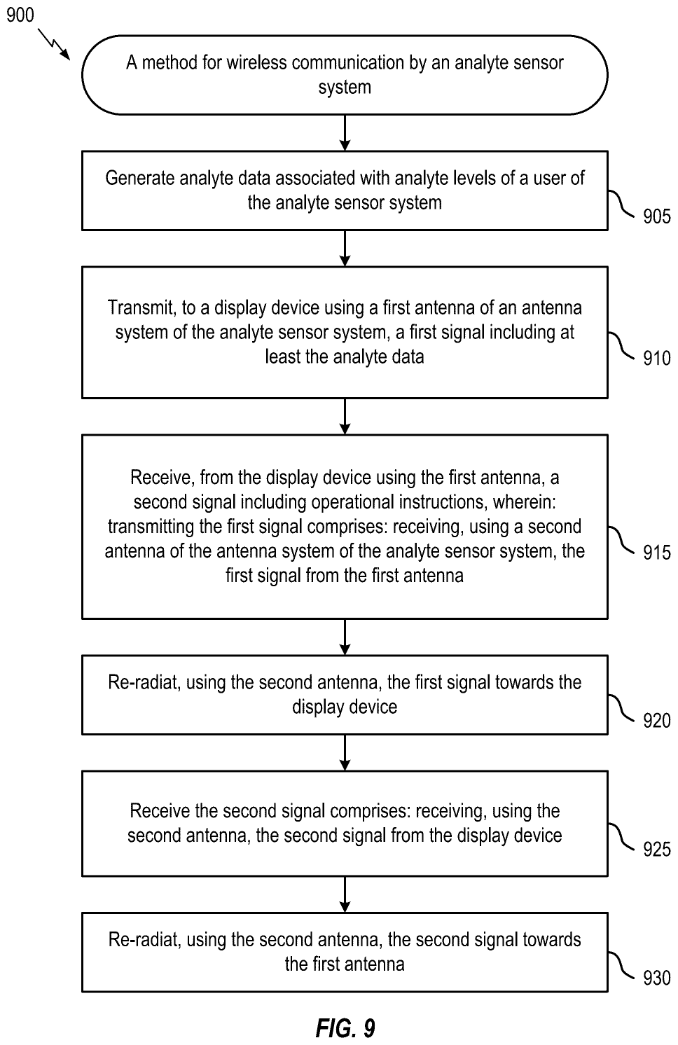

FIG. 9 shows a method 900 for wireless communications by an analyte sensor system, such as the analyte sensor system 8 depicted and described with respect to FIG. 1, the analyte sensor system 208 depicted and described with respect to FIG. 2 and FIG. 5, and/or the analyte sensor system 700 depicted and described with respect to FIG. 7.

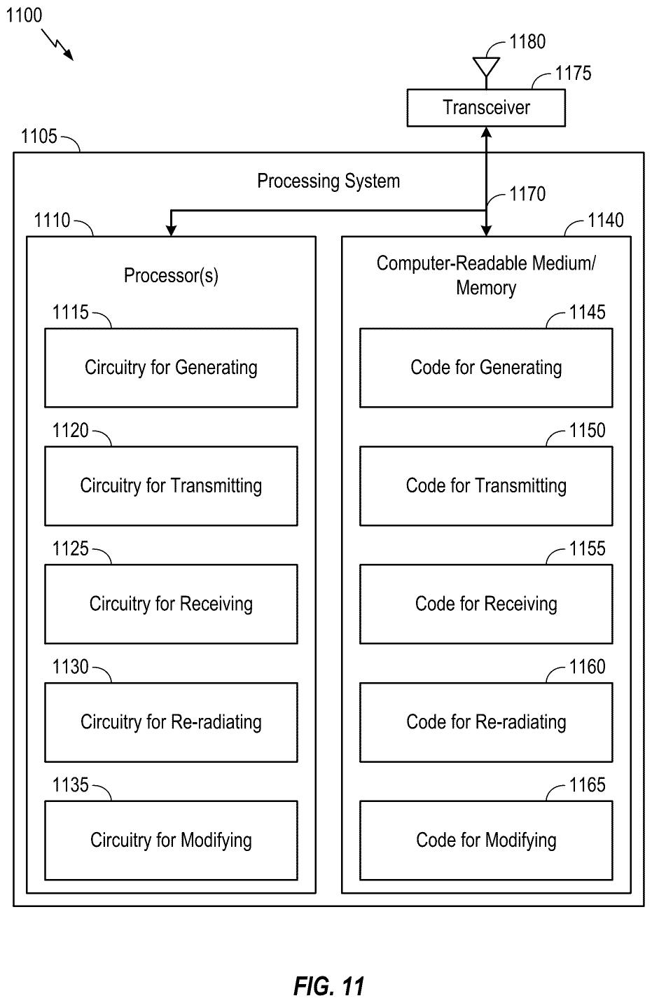

Method 900 begins at step 905 with generating analyte data associated with analyte levels of a user of the analyte sensor system. In some cases, the operations of this step refer to, or may be performed by, circuitry for generating and/or code for generating as described with reference to FIG. 11.

Method 900 then proceeds to step 910 with transmitting, to a display device using a first antenna of an antenna system of the analyte sensor system, a first signal including at least the analyte data. In some cases, the operations of this step refer to, or may be performed by, circuitry for transmitting and/or code for transmitting as described with reference to FIG. 11.

Method 900 then proceeds to step 915 with receiving, from the display device using the first antenna, a second signal including operational instructions, wherein: transmitting the first signal comprises: receiving, using a second antenna of the antenna system of the analyte sensor system, the first signal from the first antenna. In some cases, the operations of this step refer to, or may be performed by, circuitry for receiving and/or code for receiving as described with reference to FIG. 11.

Method 900 then proceeds to step 920 with re-radiating, using the second antenna, the first signal towards the display device. In some cases, the operations of this step refer to, or may be performed by, circuitry for re-radiating and/or code for re-radiating as described with reference to FIG. 11.

Method 900 then proceeds to step 925 with receiving the second signal comprises: receiving, using the second antenna, the second signal from the display device. In some cases, the operations of this step refer to, or may be performed by, circuitry for receiving and/or code for receiving as described with reference to FIG. 11.

Method 900 then proceeds to step 930 with re-radiating, using the second antenna, the second signal towards the first antenna. In some cases, the operations of this step refer to, or may be performed by, circuitry for re-radiating and/or code for re-radiating as described with reference to FIG. 11.

In some aspects, the first antenna and the circuit board are included within a housing of the analyte sensor system.

In some aspects, the second antenna is included in the housing of the analyte sensor system.

In some aspects, the second antenna is disposed outside of the housing of the analyte sensor system.

In some aspects, the second antenna is incorporated into an adhesive patch attached to an outside of the housing of the analyte sensor system.

In some aspects, the first antenna comprises a main antenna; and the second antenna comprises a passive antenna.

In some aspects, the passive antenna comprises at least one of a dipole, monopole, loop, inverted-F, or fractal antenna.

In some aspects, the passive antenna is integrated on the circuit board.

In some aspects, the passive antenna comprises portions extending off the circuit board.

In some aspects, the passive antenna extends to an outside portion of a housing of the analyte sensor system.

In some aspects, the passive antenna comprises a passive antenna terminal; and the passive antenna comprises one or more antenna arms coupled to the passive antenna terminal.

In some aspects, the one or more antenna arms comprises a plurality of arms, the plurality of arms coupled to the passive antenna terminal.

In some aspects, the passive antenna is grounded at the passive antenna terminal.

In some aspects, the passive antenna terminal comprises one or more passive electrical components.

In some aspects, the one or more passive electrical components comprise at least one of a resistor, a capacitor, or an inductor.

In some aspects, an electrical property of the one or more passive electrical components are based on at least one of a topology of the passive antenna or a distance from the passive antenna to the main antenna; and the electrical property comprises at least one of resistance, capacitance, or inductance.

In some aspects, a geometry of the passive antenna approximately matches a geometry of the main antenna.

In some aspects, a geometry of the passive antenna is configured to maximize a diagonal length from the main antenna to a portion of the passive antenna.

In some aspects, a geometry of the passive antenna is configured based on a topology to optimize communications between the main antenna and the display device.

In some aspects, a geometry of the main antenna is configured to maximize reception of the second signal.

In some aspects, the main antenna comprises at least one of a dipole, monopole, loop, inverted-F, or fractal antenna.

In some aspects, the operational instructions comprises at least one of configuration instructions, initial pairing instructions, keep alive instructions, disconnect instructions, or instructions to transmit the analyte data.

In some aspects, at least one of: the re-radiated first signal comprises a first same wavelength corresponding to the first signal; or the re-radiated second signal comprises a second same wavelength corresponding to second signal.

In some aspects, at least one of: the second antenna is configured operatively such that the re-radiated first signal comprises a first different wavelength corresponding to the first signal; or the second antenna is configured operatively such that the re-radiated second signal comprises a second different wavelength corresponding to the second signal.

In some aspects, the method 900 further includes modifying a phase shift of at least one of the first signal or the second signal. In some cases, the operations of this step refer to, or may be performed by, circuitry for modifying and/or code for modifying as described with reference to FIG. 11.

In some aspects, at least one of: a phase shift of the first signal re-radiated by the second antenna is different from a phase shift of the first signal received by the second antenna; or a phase shift of the second signal re-radiated by the second antenna is different from a phase shift of the second signal received by the second antenna.

In some aspects, the first signal, the second signal, and the re-radiated second signal are communicated according to a wireless communication technology.

In some aspects, the wireless communication technology comprises at least one of BLUETOOTH Low Energy (BLE), BLUETOOTH, or Wi-Fi.

In one aspect, method 900, or any aspect related to it, may be performed by an apparatus, such as health monitoring device 1100 of FIG. 11, which includes various components operable, configured, or adapted to perform the method 900. Health monitoring device 1100 is described below in further detail.

Note that FIG. 9 is just one example of a method, and other methods including fewer, additional, or alternative steps are possible consistent with this disclosure.

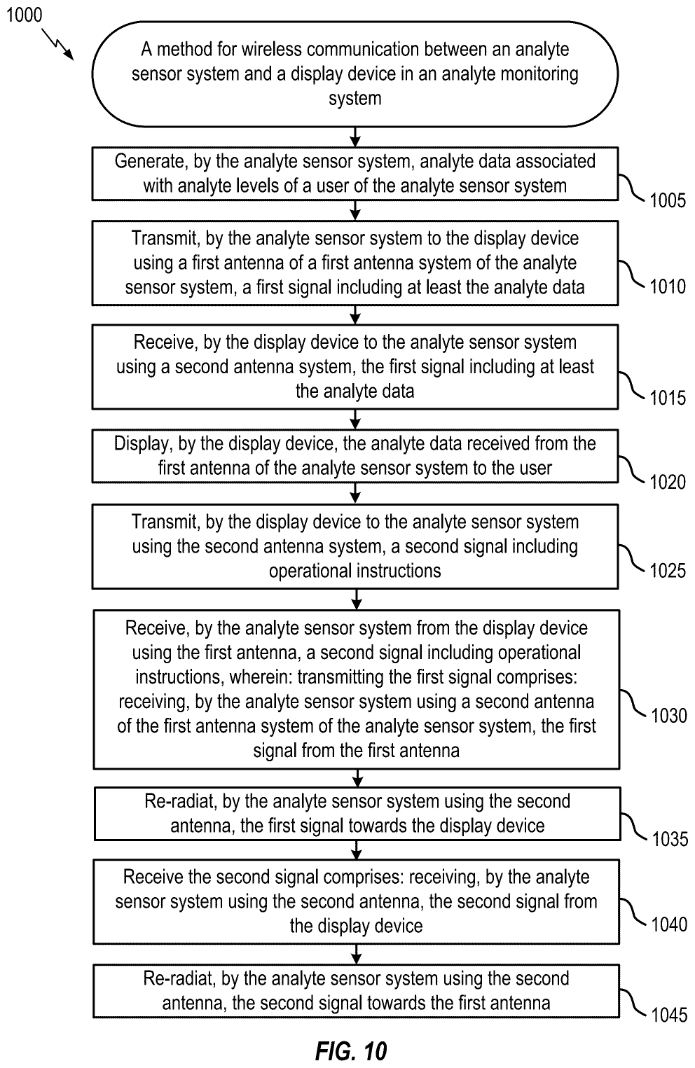

FIG. 10 shows an example of a method for communication between an analyte sensor system and a display device in an analyte monitoring system. In some embodiments, the analyte sensor system may be an example of the analyte sensor system 8 depicted and described with respect to FIG. 1, the analyte sensor system 208 depicted and described with respect to FIG. 2 and FIG. 5, and/or the analyte sensor system 700 depicted and described with respect to FIG. 7. In some embodiments, the display device may be an example of the display devices 110, 120, 130, and 140, partner devices 136, and/or server system 134 depicted and described with respect to FIG. 1 and/or the display device 210, the partner device 215, or the server system 234 depicted and described with respect to FIG. 2.

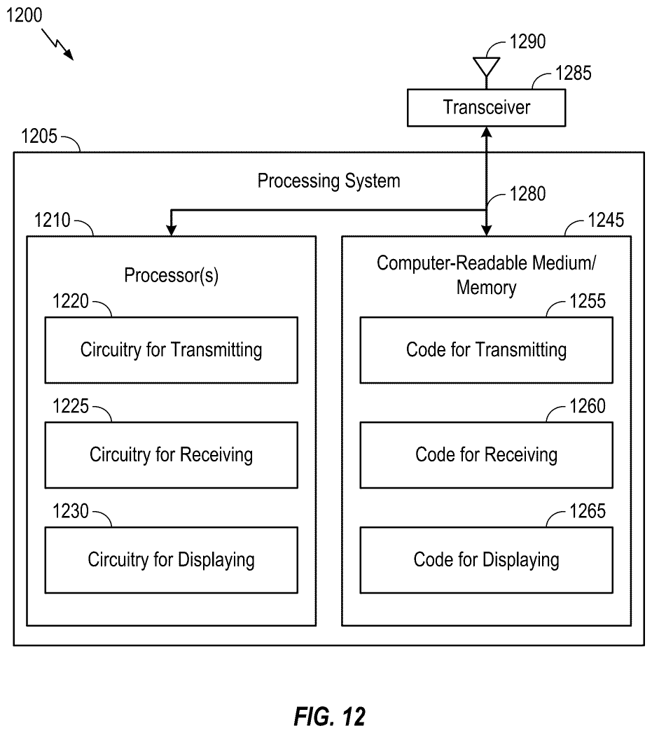

Method 1000 begins at step 1005 with generating, by the analyte sensor system, analyte data associated with analyte levels of a user of the analyte sensor system. In some cases, the operations of this step refer to, or may be performed by, circuitry for generating and/or code for generating as described with reference to FIG. 12.

Method 1000 then proceeds to step 1010 with transmitting, by the analyte sensor system to the display device using a first antenna of a first antenna system of the analyte sensor system, a first signal including at least the analyte data. In some cases, the operations of this step refer to, or may be performed by, circuitry for transmitting and/or code for transmitting as described with reference to FIG. 12.

Method 1000 then proceeds to step 1015 with receiving, by the display device to the analyte sensor system using a second antenna system, the first signal including at least the analyte data. In some cases, the operations of this step refer to, or may be performed by, circuitry for receiving and/or code for receiving as described with reference to FIG. 12.

Method 1000 then proceeds to step 1020 with displaying, by the display device, the analyte data received from the first antenna of the analyte sensor system to the user. In some cases, the operations of this step refer to, or may be performed by, circuitry for displaying and/or code for displaying as described with reference to FIG. 12.

Method 1000 then proceeds to step 1025 with transmitting, by the display device to the analyte sensor system using the second antenna system, a second signal including operational instructions. In some cases, the operations of this step refer to, or may be performed by, circuitry for transmitting and/or code for transmitting as described with reference to FIG. 12.

Method 1000 then proceeds to step 1030 with receiving, by the analyte sensor system from the display device using the first antenna, a second signal including operational instructions, wherein: transmitting the first signal comprises: receiving, by the analyte sensor system using a second antenna of the first antenna system of the analyte sensor system, the first signal from the first antenna. In some cases, the operations of this step refer to, or may be performed by, circuitry for receiving and/or code for receiving as described with reference to FIG. 12.

Method 1000 then proceeds to step 1035 with re-radiating, by the analyte sensor system using the second antenna, the first signal towards the display device. In some cases, the operations of this step refer to, or may be performed by, circuitry for re-radiating and/or code for re-radiating as described with reference to FIG. 12.

Method 1000 then proceeds to step 1040 with receiving the second signal comprises: receiving, by the analyte sensor system using the second antenna, the second signal from the display device. In some cases, the operations of this step refer to, or may be performed by, circuitry for receiving and/or code for receiving as described with reference to FIG. 12.

Method 1000 then proceeds to step 1045 with re-radiating, by the analyte sensor system using the second antenna, the second signal towards the first antenna. In some cases, the operations of this step refer to, or may be performed by, circuitry for re-radiating and/or code for re-radiating as described with reference to FIG. 12.

In some aspects, the first antenna and the circuit board are included within a housing of the analyte sensor system.

In some aspects, the second antenna is included in the housing of the analyte sensor system.

In some aspects, the second antenna is disposed outside of the housing of the analyte sensor system.

In some aspects, the second antenna is incorporated into an adhesive patch attached to an outside of the housing of the analyte sensor system.

In some aspects, the first antenna comprises a main antenna; and the second antenna comprises a passive antenna.

In some aspects, the passive antenna comprises at least one of a dipole, monopole, loop, inverted-F, or fractal antenna.

In some aspects, the passive antenna is integrated on the circuit board.

In some aspects, the passive antenna comprises portions extending off the circuit board.

In some aspects, the passive antenna extends to an outside portion of a housing of the analyte sensor system.

In some aspects, the passive antenna comprises a passive antenna terminal; and the passive antenna comprises one or more antenna arms coupled to the passive antenna terminal.

In some aspects, the one or more antenna arms comprises a plurality of arms, the plurality of arms coupled to the passive antenna terminal.

In some aspects, the passive antenna is grounded at the passive antenna terminal.

In some aspects, the passive antenna terminal comprises one or more passive electrical components.

In some aspects, the one or more passive electrical components comprise at least one of a resistor, a capacitor, or an inductor.

In some aspects, an electrical property of the one or more passive electrical components are based on at least one of a topology of the passive antenna or a distance from the passive antenna to the main antenna; and the electrical property comprises at least one of resistance, capacitance, or inductance.

In some aspects, a geometry of the passive antenna approximately matches a geometry of the main antenna.

In some aspects, a geometry of the passive antenna is configured to maximize a diagonal length from the main antenna to a portion of the passive antenna.

In some aspects, a geometry of the passive antenna is configured based on a topology to optimize communications between the main antenna and the display device.

In some aspects, a geometry of the main antenna is configured to maximize reception of the second signal.

In some aspects, the main antenna comprises at least one of a dipole, monopole, loop, inverted-F, or fractal antenna.

In some aspects, the operational instructions comprises at least one of configuration instructions, initial pairing instructions, keep alive instructions, disconnect instructions, or instructions to transmit the analyte data.

In some aspects, at least one of: the re-radiated first signal comprises a first same wavelength corresponding to the first signal; or the re-radiated second signal comprises a second same wavelength corresponding to second signal.

In some aspects, at least one of: the second antenna is configured operatively such that the re-radiated first signal comprises a first different wavelength corresponding to the first signal; or the second antenna is configured operatively such that the re-radiated second signal comprises a second different wavelength corresponding to the second signal.

In some aspects, the method 1000 further includes modifying a phase shift of at least one of the first signal or the second signal. In some cases, the operations of this step refer to, or may be performed by, circuitry for modifying and/or code for modifying as described with reference to FIG. 12.

In some aspects, at least one of: a phase shift of the first signal re-radiated by the second antenna is different from a phase shift of the first signal received by the second antenna; or a phase shift of the second signal re-radiated by the second antenna is different from a phase shift of the second signal received by the second antenna.

In some aspects, the first signal, the second signal, and the re-radiated second signal are communicated according to a wireless communication technology.

In some aspects, the wireless communication technology comprises at least one of BLUETOOTH Low Energy (BLE), BLUETOOTH, or Wi-Fi.

In one aspect, method 1000, or any aspect related to it, may be performed by an apparatus, such as health monitoring device 1200 of FIG. 12, which includes various components operable, configured, or adapted to perform the method 1000. Health monitoring device 1200 is described below in further detail.

Note that FIG. 10 is just one example of a method, and other methods including fewer, additional, or alternative steps are possible consistent with this disclosure.

Example Communications Device(s)

FIG. 11 depicts aspects of an example health monitoring device 1100. In some aspects, health monitoring device 1100 is an analyte sensor system, such as the analyte sensor system 8 described with respect to FIGS. 1, the analyte sensor system 208 of FIGS. 2 and 5, and/or the analyte sensor system 700 of FIG. 7.

The health monitoring device 1100 includes a processing system 1105 coupled to the transceiver 1175 (e.g., a transmitter and/or a receiver). The transceiver 1175 is configured to transmit and receive signals for the health monitoring device 1100 via the first antenna system 1180, such as the various signals as described herein. The processing system 1105 may be configured to perform processing functions for the health monitoring device 1100, including processing signals received and/or to be transmitted by the health monitoring device 1100.

The processing system 1105 includes one or more processors 1110. In various aspects, the one or more processors 1310 may be representative of the processor/microcontroller 535, as described with respect to FIG. 5. The one or more processors 1110 are coupled to a computer-readable medium/memory 1140 via a bus 1170. In some aspects, the computer-readable medium/memory 1345 may be representative of the storage 515, as described with respect to FIG. 5. In certain aspects, the computer-readable medium/memory 1140 is configured to store instructions (e.g., computer-executable code) that when executed by the one or more processors 1110, cause the one or more processors 1110 to perform the methods 900 and/or 1000 described with respect to FIGS. 9 and 10, or any aspects related to these methods. Note that reference to a processor performing a function of health monitoring device 1100 may include one or more processors 1110 performing that function of health monitoring device 1100.

In the depicted example, computer-readable medium/memory 1140 stores code (e.g., executable instructions), such as code for generating 1145, code for transmitting 1150, code for receiving 1155, code for re-radiating 1160, and code for modifying 1165. Processing of the code for generating 1145, code for transmitting 1150, code for receiving 1155, code for re-radiating 1160, and code for modifying 1165 may cause the health monitoring device 1100 to perform the methods 900 and/or 1000 described with respect to FIGS. 9 and 10, or any aspects related to these methods.

The one or more processors 1110 include circuitry configured to implement (e.g., execute) the code stored in the computer-readable medium/memory 1140, including circuitry such as circuitry for generating 1115, circuitry for transmitting 1120, circuitry for receiving 1125, circuitry for re-radiating 1130, and circuitry for modifying 1135. Processing with circuitry for generating 1115, circuitry for transmitting 1120, circuitry for receiving 1125, circuitry for re-radiating 1130, and circuitry for modifying 1135 may cause the health monitoring device 1100 to perform the methods 900 and/or 1000 described with respect to FIGS. 9 and 10, or any aspects related to these methods.

FIG. 12 depicts aspects of an example health monitoring device 1200. In some aspects, health monitoring device 1200 is a display device, such as display devices 110, 120, 130, and 140, partner devices 136, and/or server system 134 depicted and described with respect to FIG. 1 and/or the display device 210, the partner device 215, or the server system 234 depicted and described with respect to FIG. 2.

The health monitoring device 1200 includes a processing system 1205 coupled to the transceiver 1285 (e.g., a transmitter and/or a receiver). The transceiver 1285 is configured to transmit and receive signals for the health monitoring device 1200 via the antenna 1290, such as the various signals as described herein. The processing system 1205 may be configured to perform processing functions for the health monitoring device 1200, including processing signals received and/or to be transmitted by the health monitoring device 1200.

The processing system 1205 includes one or more processors 1210. The one or more processors 1210 are coupled to a computer-readable medium/memory 1245 via a bus 1280. In certain aspects, the computer-readable medium/memory 1245 is configured to store instructions (e.g., computer-executable code) that when executed by the one or more processors 1210, cause the one or more processors 1210 to perform the method 1000 described with respect to FIG. 10, or any aspect related to it. Note that reference to a processor performing a function of health monitoring device 1200 may include one or more processors 1210 performing that function of health monitoring device 1200.

In the depicted example, computer-readable medium/memory 1245 stores code (e.g., executable instructions), such as code for transmitting 1255, code for receiving 1260, and code for displaying 1265. Processing of the code for transmitting 1255, code for receiving 1260, and code for displaying 1265 may cause the health monitoring device 1200 to perform the method 1000 described with respect to FIG. 10, or any aspect related to it.

The one or more processors 1210 include circuitry configured to implement (e.g., execute) the code stored in the computer-readable medium/memory 1245, including circuitry such as circuitry for transmitting 1220, circuitry for receiving 1225, and circuitry for displaying 1230. Processing with circuitry for transmitting 1220, circuitry for receiving 1225, and circuitry for displaying 1230 may cause the health monitoring device 1200 to perform the method 1000 described with respect to FIG. 10, or any aspect related to it.

### Example Clauses

Implementation examples are described in the following numbered clauses:

Clause 1: An analyte sensor system, comprising: an analyte sensor configured to generate analyte data associated with analyte levels of a user of the analyte sensor system; an antenna system, comprising at least a first antenna and a second antenna, wherein: the first antenna is configured to: transmit, to a display device, a first signal including at least the analyte data; and receive, from the display device, a second signal including operational instructions; the second antenna is configured to: receive the first signal from the first antenna and re-radiate the first signal towards the display device; and receive the second signal from the display device and re-radiate the second signal towards the first antenna; and a circuit board configured to operatively connect the analyte sensor with the first antenna of the antenna system.

Clause 2: The analyte sensor system of Clause 1, wherein the first antenna and the circuit board are included within a housing of the analyte sensor system.

Clause 3: The analyte sensor system of Clause 2, wherein the second antenna is included in the housing of the analyte sensor system.

Clause 4: The analyte sensor system of Clause 2, wherein the second antenna is disposed outside of the housing of the analyte sensor system.

Clause 5: The analyte sensor system of Clause 4, wherein the second antenna is incorporated into an adhesive patch attached to an outside of the housing of the analyte sensor system.

Clause 6: The analyte sensor system of any one of Clauses 1-5, wherein: the first antenna comprises a main antenna; and the second antenna comprises a passive antenna.

Clause 7: The analyte sensor system of Clause 6, wherein the passive antenna comprises at least one of a dipole, monopole, loop, inverted-F, or fractal antenna.

Clause 8: The analyte sensor system of any one of Clauses 6-7, wherein the passive antenna is integrated on the circuit board.

Clause 9: The analyte sensor system of any one of Clauses 6-7, wherein the passive antenna comprises portions extending off the circuit board.

Clause 10: The analyte sensor of Clause 9, wherein the passive antenna extends to an outside portion of a housing of the analyte sensor system.

Clause 11: The analyte sensor system of any one of Clauses 6-10, wherein: the passive antenna comprises a passive antenna terminal; and the passive antenna comprises one or more antenna arms coupled to the passive antenna terminal.

Clause 12: The analyte sensor system of Clause 11, wherein the one or more antenna arms comprises a plurality of arms, the plurality of arms coupled to the passive antenna terminal.

Clause 13: The analyte sensor system of any one of Clauses 11-12, wherein the passive antenna is grounded at the passive antenna terminal.

Clause 14: The analyte sensor system of any one of Clauses 11-13, wherein the passive antenna terminal comprises one or more passive electrical components.

Clause 15: The analyte sensor system of Clause 14, wherein the one or more passive electrical components comprise at least one of a resistor, a capacitor, or an inductor.

Clause 16: The analyte sensor system of any one of Clauses 14-15, wherein: an electrical property of the one or more passive electrical components are based on at least one of a topology of the passive antenna or a distance from the passive antenna to the main antenna; and the electrical property comprises at least one of resistance, capacitance, or inductance.

Clause 17: The analyte sensor system of any one of Clauses 6-16, wherein a geometry of the passive antenna approximately matches a geometry of the main antenna.

Clause 18: The analyte sensor system of any one of Clauses 6-16, wherein a geometry of the passive antenna is configured to maximize a diagonal length from the main antenna to a portion of the passive antenna.

Clause 19: The analyte sensor system of any one of Clauses 6-16, wherein a geometry of the passive antenna is configured based on a topology to optimize communications between the main antenna and the display device.

Clause 20: The analyte sensor system of any one of Clauses 6-19, wherein a geometry of the main antenna is configured to maximize reception of the second signal.

Clause 21: The analyte sensor system of any one of Clauses 6-20, wherein the main antenna comprises at least one of a dipole, monopole, loop, inverted-F, or fractal antenna.

Clause 22: The analyte sensor system of any one of Clauses 1-21, wherein the operational instructions comprises at least one of configuration instructions, initial pairing instructions, keep alive instructions, disconnect instructions, or instructions to transmit the analyte data.

Clause 23: The analyte sensor system of any one of Clauses 1-22, wherein at least one of: the re-radiated first signal comprises a first same wavelength corresponding to the first signal; or the re-radiated second signal comprises a second same wavelength corresponding to second signal.

Clause 24: The analyte sensor system of any one of Clauses 1-22, wherein at least one of: the second antenna is configured operatively such that the re-radiated first signal comprises a first different wavelength corresponding to the first signal; or the second antenna is configured operatively such that the re-radiated second signal comprises a second different wavelength corresponding to the second signal.

Clause 25: The analyte sensor system any one of Clauses 1-24, wherein the second antenna is configured to modify a phase shift of at least one of the first signal or the second signal.

Clause 26: The analyte sensor system of Clause 25, wherein at least one of: a phase shift of the first signal re-radiated by the second antenna is different from a phase shift of the first signal received by the second antenna; or a phase shift of the second signal re-radiated by the second antenna is different from a phase shift of the second signal received by the second antenna.

Clause 27: The analyte sensor system of any one of Clauses 1-26, further comprising a transceiver, coupled to the first antenna, configured to: transmit the first signal; and receive at least one of the second signal from the display device or the re-radiated signal from the second antenna.

Clause 28: The analyte sensor system of Clause 27, wherein the transceiver is configured to communicate signals, including the first signal, the second signal, and the re-radiated second signal, according to a wireless communication technology.

Clause 29: The analyte sensor system of Clause 28, wherein the wireless communication technology comprises at least one of BLUETOOTH Low Energy (BLE), BLUETOOTH, or Wi-Fi.

Clause 30: The analyte sensor system of any one of Clauses 1-29, further comprising one or more processors and one or more memories, wherein the circuit board is further configured to operatively connect the one or more processors and one or more memories to the analyte sensor, the first antenna, and a transceiver.

Clause 31: The analyte sensor system of Clause 30, wherein the one or more processors are configured to: obtain and process the analyte data from the analyte sensor; and provide the processed analyte data to the transceiver for transmission via the first antenna.

Clause 32: An antenna system for communicating analyte data, comprising: a first antenna operatively coupled to an analyte sensor via a circuit board, wherein the first antenna is configured to: transmit, to a display device, a first signal including at least the analyte data; and receive, from the display device, a second signal including operational instructions; and a second antenna configured to: receive the first signal from the first antenna and re-radiate the first signal towards the display device; and receive the second signal from the display device and re-radiate the second signal towards the first antenna.

Clause 33: The antenna system of Clause 32, wherein the first antenna and the circuit board are included within a housing of the antenna system.

Clause 34: The antenna system of Clause 33, wherein the second antenna is included in the housing of the antenna system.

Clause 35: The antenna system of Clause 33, wherein the second antenna is disposed outside of the housing of the antenna system.

Clause 36: The antenna system of Clause 35, wherein the second antenna is incorporated into an adhesive patch attached to an outside of the housing of the antenna system.

Clause 37: The antenna system of any one of Clauses 32-36, wherein: the first antenna comprises a main antenna; and the second antenna comprises a passive antenna.

Clause 38: The antenna system of Clause 37, wherein the passive antenna comprises at least one of a dipole, monopole, loop, inverted-F, or fractal antenna.

Clause 39: The antenna system of any one of Clauses 37-38, wherein the passive antenna is integrated on the circuit board.

Clause 40: The antenna system of any one of Clauses 37-39, wherein the passive antenna comprises portions extending off the circuit board.

Clause 41: The analyte sensor of Clause 40, wherein the passive antenna extends to an outside portion of a housing of the antenna system.

Clause 42: The antenna system of any one of Clauses 37-41, wherein: the passive antenna comprises a passive antenna terminal; and the passive antenna comprises one or more antenna arms coupled to the passive antenna terminal.

Clause 43: The antenna system of Clause 42, wherein the one or more antenna arms comprises a plurality of arms, the plurality of arms coupled to the passive antenna terminal.

Clause 44: The antenna system of any one of Clauses 42-43, wherein the passive antenna is grounded at the passive antenna terminal.

Clause 45: The antenna system of any one of Clauses 42-44, wherein the passive antenna terminal comprises one or more passive electrical components.

Clause 46: The antenna system of Clause 45, wherein the one or more passive electrical components comprise at least one of a resistor, a capacitor, or an inductor.

Clause 47: The antenna system of any one of Clauses 45-46, wherein: an electrical property of the one or more passive electrical components are based on at least one of a topology of the passive antenna or a distance from the passive antenna to the main antenna; and the electrical property comprises at least one of resistance, capacitance, or inductance.

Clause 48: The antenna system of any one of Clauses 37-47, wherein a geometry of the passive antenna approximately matches a geometry of the main antenna.

Clause 49: The antenna system of any one of Clauses 37-47, wherein a geometry of the passive antenna is configured to maximize a diagonal length from the main antenna to a portion of the passive antenna.

Clause 50: The antenna system of any one of Clauses 37-47, wherein a geometry of the passive antenna is configured based on a topology to optimize communications between the main antenna and the display device.

Clause 51: The antenna system of any one of Clauses 37-50, wherein a geometry of the main antenna is configured to maximize reception of the second signal.

Clause 52: The antenna system of any one of Clauses 37-51, wherein the main antenna comprises at least one of a dipole, monopole, loop, inverted-F, or fractal antenna.

Clause 53: The antenna system of any one of Clauses 32-52, wherein the operational instructions comprises at least one of configuration instructions, initial pairing instructions, keep alive instructions, disconnect instructions, or instructions to transmit the analyte data.

Clause 54: The antenna system of any one of Clauses 32-53, wherein at least on of: the re-radiated first signal comprises a first same wavelength corresponding to the first signal; or the re-radiated second signal comprises a second same wavelength corresponding to second signal.

Clause 55: The antenna system of any one of Clauses 32-53, wherein at least one of: the second antenna is configured operatively such that the re-radiated first signal comprises a first different wavelength corresponding to the first signal; or the second antenna is configured operatively such that the re-radiated second signal comprises a second different wavelength corresponding to the second signal.

Clause 56: The antenna system of any one of Clauses 32-55, wherein the second antenna is configured to modify a phase shift of at least one of the first signal or the second signal.

Clause 57: The antenna system of Clause 56, wherein at least one of: a phase shift of the first signal re-radiated by the second antenna is different from a phase shift of the first signal received by the second antenna; or a phase shift of the second signal re-radiated by the second antenna is different from a phase shift of the second signal received by the second antenna.

Clause 58: The antenna system of any one of Clauses 32-57, further comprising a transceiver, coupled to the first antenna, configured to: transmit the first signal; and receive at least one of the second signal from the display device or the re-radiated signal from the second antenna.

Clause 59: The antenna system of Clause 58, wherein the transceiver is configured to communicate signals, including the first signal, the second signal, and the re-radiated second signal, according to a wireless communication technology.

Clause 60: The antenna system of Clause 59, wherein the wireless communication technology comprises at least one of BLUETOOTH Low Energy (BLE), BLUETOOTH, or Wi-Fi.

Clause 61: The antenna system of any one of Clauses 32-60, further comprising one or more processors and one or more memories, wherein the circuit board is further configured to operatively connect the one or more processors and one or more memories to the analyte sensor, the first antenna, and a transceiver.

Clause 62: The antenna system of Clause 61, wherein the one or more processors are configured to: obtain and process the analyte data from the analyte sensor; and provide the processed analyte data to the transceiver for transmission via the first antenna.

Clause 63: An analyte monitoring system, comprising: a display device; and an analyte sensor system comprising: an analyte sensor configured to generate analyte data associated with analyte levels of a user of the analyte sensor system; a first antenna configured to: transmit, to the display device, a first signal including at least the analyte data; and receive, from the display device, a second signal including operational instructions; a second antenna configured to: receive the first signal from the first antenna and re-radiate the first signal towards the display device; and receive the second signal from the display device and re-radiate the second signal towards the first antenna; and a circuit board configured to operatively connect the analyte sensor with the first antenna, wherein the display device is configured to display the analyte data received from the first antenna of the analyte sensor system to the user.

Clause 64: The analyte monitoring system of Clause 63, wherein the first antenna and the circuit board are included within a housing of the analyte monitoring system.

Clause 65: The analyte monitoring system of Clause 64, wherein the second antenna is included in the housing of the analyte monitoring system.

Clause 66: The analyte monitoring system of Clause 64, wherein the second antenna is disposed outside of the housing of the analyte monitoring system.

Clause 67: The analyte monitoring system of Clause 66, wherein the second antenna is incorporated into an adhesive patch attached to an outside of the housing of the analyte monitoring system.

Clause 68: The analyte monitoring system of any one of Clauses 63-67, wherein: the first antenna comprises a main antenna; and the second antenna comprises a passive antenna.

Clause 69: The analyte monitoring system of Clause 68, wherein the passive antenna comprises at least one of a dipole, monopole, loop, inverted-F, or fractal antenna.

Clause 70: The analyte monitoring system of any one of Clauses 68-69, wherein the passive antenna is integrated on the circuit board.

Clause 71: The analyte monitoring system of any one of Clauses 68-70, wherein the passive antenna comprises portions extending off the circuit board.

Clause 72: The analyte sensor of Clause 71, wherein the passive antenna extends to an outside portion of a housing of the analyte monitoring system.

Clause 73: The analyte monitoring system of any one of Clauses 68-72, wherein: the passive antenna comprises a passive antenna terminal; and the passive antenna comprises one or more antenna arms coupled to the passive antenna terminal.

Clause 74: The analyte monitoring system of Clause 73, wherein the one or more antenna arms comprises a plurality of arms, the plurality of arms coupled to the passive antenna terminal.

Clause 75: The analyte monitoring system of any one of Clauses 73-74, wherein the passive antenna is grounded at the passive antenna terminal.

Clause 76: The analyte monitoring system of any one of Clauses 73-75, wherein the passive antenna terminal comprises one or more passive electrical components.

Clause 77: The analyte monitoring system of Clause 76, wherein the one or more passive electrical components comprise at least one of a resistor, a capacitor, or an inductor.

Clause 78: The analyte monitoring system of any one of Clauses 76-77, wherein: an electrical property of the one or more passive electrical components are based on at least one of a topology of the passive antenna or a distance from the passive antenna to the main antenna; and the electrical property comprises at least one of resistance, capacitance, or inductance.

Clause 79: The analyte monitoring system of any one of Clauses 68-78, wherein a geometry of the passive antenna approximately matches a geometry of the main antenna.

Clause 80: The analyte monitoring system of any one of Clauses 68-78, wherein a geometry of the passive antenna is configured to maximize a diagonal length from the main antenna to a portion of the passive antenna.

Clause 81: The analyte monitoring system of any one of Clauses 68-78, wherein a geometry of the passive antenna is configured based on a topology to optimize communications between the main antenna and the display device.

Clause 82: The analyte monitoring system of any one of Clauses 68-81, wherein a geometry of the main antenna is configured to maximize reception of the second signal.

Clause 83: The analyte monitoring system of any one of Clauses 68-82, wherein the main antenna comprises at least one of a dipole, monopole, loop, inverted-F, or fractal antenna.

Clause 84: The analyte monitoring system of any one of Clauses 63-83, wherein the operational instructions comprises at least one of configuration instructions, initial pairing instructions, keep alive instructions, disconnect instructions, or instructions to transmit the analyte data.

Clause 85: The analyte monitoring system of any one of Clauses 63-84, wherein at least one of: the re-radiated first signal comprises a first same wavelength corresponding to the first signal; or the re-radiated second signal comprises a second same wavelength corresponding to second signal.

Clause 86: The analyte monitoring system of any one of Clauses 63-84, wherein at least one of: the second antenna is configured operatively such that the re-radiated first signal comprises a first different wavelength corresponding to the first signal; or the second antenna is configured operatively such that the re-radiated second signal comprises a second different wavelength corresponding to the second signal.

Clause 87: The analyte monitoring system of any one of Clauses 63-86, wherein the second antenna is configured to modify a phase shift of at least one of the first signal or the second signal.

Clause 88: The analyte monitoring system of Clause 87, wherein at least one of: a phase shift of the first signal re-radiated by the second antenna is different from a phase shift of the first signal received by the second antenna; or a phase shift of the second signal re-radiated by the second antenna is different from a phase shift of the second signal received by the second antenna.

Clause 89: The analyte monitoring system of any one of Clauses 63-88, further comprising a transceiver, coupled to the first antenna, configured to: transmit the first signal; and receive at least one of the second signal from the display device or the re-radiated signal from the second antenna.

Clause 90: The analyte monitoring system of Clause 89, wherein the transceiver is configured to communicate signals, including the first signal, the second signal, and the re-radiated second signal, according to a wireless communication technology.

Clause 91: The analyte monitoring system of Clause 90, wherein the wireless communication technology comprises at least one of BLUETOOTH Low Energy (BLE), BLUETOOTH, or Wi-Fi.

Clause 92: The analyte monitoring system of any one of Clauses 63-91, further comprising one or more processors and one or more memories, wherein the circuit board is further configured to operatively connect the one or more processors and one or more memories to the analyte sensor, the first antenna, and a transceiver.

Clause 93: The analyte monitoring system of Clause 92, wherein the one or more processors are configured to: obtain and process the analyte data from the analyte sensor; and provide the processed analyte data to the transceiver for transmission via the first antenna.

Clause 94: A method for wireless communication by an analyte sensor system, comprising: generating analyte data associated with analyte levels of a user of the analyte sensor system; transmitting, to a display device using a first antenna of an antenna system of the analyte sensor system, a first signal including at least the analyte data; receiving, from the display device using the first antenna, a second signal including operational instructions, wherein: transmitting the first signal comprises: receiving, using a second antenna of the antenna system of the analyte sensor system, the first signal from the first antenna; re-radiating, using the second antenna, the first signal towards the display device; receiving the second signal comprises: receiving, using the second antenna, the second signal from the display device; and re-radiating, using the second antenna, the second signal towards the first antenna.

Clause 95: The method of Clause 94, wherein the first antenna and the circuit board are included within a housing of the analyte sensor system.

Clause 96: The method of Clause 95, wherein the second antenna is included in the housing of the analyte sensor system.

Clause 97: The method of Clause 95, wherein the second antenna is disposed outside of the housing of the analyte sensor system.

Clause 98: The method of Clause 97, wherein the second antenna is incorporated into an adhesive patch attached to an outside of the housing of the analyte sensor system.

Clause 99: The method of any one of Clauses 94-98, wherein: the first antenna comprises a main antenna; and the second antenna comprises a passive antenna.

Clause 100: The method of Clause 99, wherein the passive antenna comprises at least one of a dipole, monopole, loop, inverted-F, or fractal antenna.

Clause 101: The method of Clause 99, wherein the passive antenna is integrated on the circuit board.

Clause 102: The method of Clause 99, wherein the passive antenna comprises portions extending off the circuit board.

Clause 103: The method of Clause 102, wherein the passive antenna extends to an outside portion of a housing of the analyte sensor system.

Clause 104: The method of Clause 99, wherein: the passive antenna comprises a passive antenna terminal; and the passive antenna comprises one or more antenna arms coupled to the passive antenna terminal.

Clause 105: The method of Clause 104, wherein the one or more antenna arms comprises a plurality of arms, the plurality of arms coupled to the passive antenna terminal.

Clause 106: The method of Clause 104, wherein the passive antenna is grounded at the passive antenna terminal.

Clause 107: The method of Clause 104, wherein the passive antenna terminal comprises one or more passive electrical components.

Clause 108: The method of Clause 107, wherein the one or more passive electrical components comprise at least one of a resistor, a capacitor, or an inductor.

Clause 109: The method of Clause 107, wherein: an electrical property of the one or more passive electrical components are based on at least one of a topology of the passive antenna or a distance from the passive antenna to the main antenna; and the electrical property comprises at least one of resistance, capacitance, or inductance.

Clause 110: The method of Clause 99, wherein a geometry of the passive antenna approximately matches a geometry of the main antenna.

Clause 111: The method of Clause 99, wherein a geometry of the passive antenna is configured to maximize a diagonal length from the main antenna to a portion of the passive antenna.

Clause 112: The method of Clause 99, wherein a geometry of the passive antenna is configured based on a topology to optimize communications between the main antenna and the display device.

Clause 113: The method of Clause 99, wherein a geometry of the main antenna is configured to maximize reception of the second signal.

Clause 114: The method of Clause 99, wherein the main antenna comprises at least one of a dipole, monopole, loop, inverted-F, or fractal antenna.

Clause 115: The method of any one of Clauses 94-114, wherein the operational instructions comprises at least one of configuration instructions, initial pairing instructions, keep alive instructions, disconnect instructions, or instructions to transmit the analyte data.

Clause 116: The method of any one of Clauses 94-115, wherein at least one of: the re-radiated first signal comprises a first same wavelength corresponding to the first signal; or the re-radiated second signal comprises a second same wavelength corresponding to second signal.

Clause 117: The method of any one of Clauses 94-116, wherein at least one of: the second antenna is configured operatively such that the re-radiated first signal comprises a first different wavelength corresponding to the first signal; or the second antenna is configured operatively such that the re-radiated second signal comprises a second different wavelength corresponding to the second signal.

Clause 118: The method of any one of Clauses 94-116, further comprising modifying a phase shift of at least one of the first signal or the second signal.

Clause 119: The method of Clause 118, wherein at least one of: a phase shift of the first signal re-radiated by the second antenna is different from a phase shift of the first signal received by the second antenna; or a phase shift of the second signal re-radiated by the second antenna is different from a phase shift of the second signal received by the second antenna.

Clause 120: The method of any one of Clauses 94-119, wherein the first signal, the second signal, and the re-radiated second signal are communicated according to a wireless communication technology.

Clause 121: The method of Clause 120, wherein the wireless communication technology comprises at least one of BLUETOOTH Low Energy (BLE), BLUETOOTH, or Wi-Fi.

Clause 122: A method for wireless communication between an analyte sensor system and a display device in an analyte monitoring system, comprising: generating, by the analyte sensor system, analyte data associated with analyte levels of a user of the analyte sensor system; transmitting, by the analyte sensor system to the display device using a first antenna of a first antenna system of the analyte sensor system, a first signal including at least the analyte data; receiving, by the display device to the analyte sensor system using a second antenna system, the first signal including at least the analyte data; displaying, by the display device, the analyte data received from the first antenna of the analyte sensor system to the user; transmitting, by the display device to the analyte sensor system using the second antenna system, a second signal including operational instructions; receiving, by the analyte sensor system from the display device using the first antenna, a second signal including operational instructions, wherein: transmitting the first signal comprises: receiving, by the analyte sensor system using a second antenna of the first antenna system of the analyte sensor system, the first signal from the first antenna; re-radiating, by the analyte sensor system using the second antenna, the first signal towards the display device; receiving the second signal comprises: receiving, by the analyte sensor system using the second antenna, the second signal from the display device; and re-radiating, by the analyte sensor system using the second antenna, the second signal towards the first antenna.

Clause 123: The method of Clause 122, wherein the first antenna and the circuit board are included within a housing of the analyte sensor system.

Clause 124: The method of Clause 123, wherein the second antenna is included in the housing of the analyte sensor system.

Clause 125: The method of Clause 123, wherein the second antenna is disposed outside of the housing of the analyte sensor system.

Clause 126: The method of Clause 125, wherein the second antenna is incorporated into an adhesive patch attached to an outside of the housing of the analyte sensor system.

Clause 127: The method of any one of Clauses 122-126, wherein: the first antenna comprises a main antenna; and the second antenna comprises a passive antenna.

Clause 128: The method of Clause 127, wherein the passive antenna comprises at least one of a dipole, monopole, loop, inverted-F, or fractal antenna.

Clause 129: The method of Clause 127, wherein the passive antenna is integrated on the circuit board.

Clause 130: The method of Clause 127, wherein the passive antenna comprises portions extending off the circuit board.

Clause 131: The method of Clause 130, wherein the passive antenna extends to an outside portion of a housing of the analyte sensor system.

Clause 132: The method of Clause 127, wherein: the passive antenna comprises a passive antenna terminal; and the passive antenna comprises one or more antenna arms coupled to the passive antenna terminal.

Clause 133: The method of Clause 132, wherein the one or more antenna arms comprises a plurality of arms, the plurality of arms coupled to the passive antenna terminal.

Clause 134: The method of Clause 132, wherein the passive antenna is grounded at the passive antenna terminal.

Clause 135: The method of Clause 132, wherein the passive antenna terminal comprises one or more passive electrical components.

Clause 136: The method of Clause 135, wherein the one or more passive electrical components comprise at least one of a resistor, a capacitor, or an inductor.

Clause 137: The method of Clause 135, wherein: an electrical property of the one or more passive electrical components are based on at least one of a topology of the passive antenna or a distance from the passive antenna to the main antenna; and the electrical property comprises at least one of resistance, capacitance, or inductance.

Clause 138: The method of Clause 127, wherein a geometry of the passive antenna approximately matches a geometry of the main antenna.

Clause 139: The method of Clause 127, wherein a geometry of the passive antenna is configured to maximize a diagonal length from the main antenna to a portion of the passive antenna.

Clause 140: The method of Clause 127, wherein a geometry of the passive antenna is configured based on a topology to optimize communications between the main antenna and the display device.

Clause 141: The method of Clause 127, wherein a geometry of the main antenna is configured to maximize reception of the second signal.

Clause 142: The method of Clause 127, wherein the main antenna comprises at least one of a dipole, monopole, loop, inverted-F, or fractal antenna.

Clause 143: The method of any one of Clauses 122-142, wherein the operational instructions comprises at least one of configuration instructions, initial pairing instructions, keep alive instructions, disconnect instructions, or instructions to transmit the analyte data.

Clause 144: The method of any one of Clauses 122-143, wherein at least one of: the re-radiated first signal comprises a first same wavelength corresponding to the first signal; or the re-radiated second signal comprises a second same wavelength corresponding to second signal.

Clause 145: The method of any one of Clauses 122-144, wherein at least one of: the second antenna is configured operatively such that the re-radiated first signal comprises a first different wavelength corresponding to the first signal; or the second antenna is configured operatively such that the re-radiated second signal comprises a second different wavelength corresponding to the second signal.

Clause 146: The method of any one of Clauses 122-145, further comprising modifying a phase shift of at least one of the first signal or the second signal.

Clause 147: The method of Clause 146, wherein at least one of: a phase shift of the first signal re-radiated by the second antenna is different from a phase shift of the first signal received by the second antenna; or a phase shift of the second signal re-radiated by the second antenna is different from a phase shift of the second signal received by the second antenna.

Clause 148: The method of any one of Clauses 122-147, wherein the first signal, the second signal, and the re-radiated second signal are communicated according to a wireless communication technology.

Clause 149: The method of Clause 148, wherein the wireless communication technology comprises at least one of BLUETOOTH Low Energy (BLE), BLUETOOTH, or Wi-Fi.

Clause 150: An apparatus, comprising: at least one memory comprising executable instructions; and at least one processor configured to execute the executable instructions and cause the apparatus to perform a method in accordance with any combination of Clauses 94-149.

Clause 151: An apparatus, comprising means for performing a method in accordance with any combination of Clauses 94-149.

Clause 152: A non-transitory computer-readable medium comprising executable instructions that, when executed by at least one processor of an apparatus, cause the apparatus to perform a method in accordance with any combination of Clauses 94-149.

Clause 153: A computer program product embodied on a computer-readable storage medium comprising code for performing a method in accordance with any combination of Clauses 94-149.

### Additional Considerations

In this document, the terms “computer program medium” and “computer usable medium” and “computer readable medium”, as well as variations thereof, are used to generally refer to transitory or non-transitory media. These and other various forms of computer program media or computer usable/readable media may be involved in carrying one or more sequences of one or more instructions to a processing device for execution. Such instructions embodied on the medium, may generally be referred to as “computer program code” or a “computer program product” or “instructions” (which may be grouped in the form of computer programs or other groupings). When executed, such instructions may enable a computing module, such as the analyte sensor system 208, analyte sensor system 600, and/or analyte sensor system 700, circuitry related thereto, and/or a processor thereof or connected thereto to perform features or functions of the present disclosure as discussed herein (for example, in connection with methods described above and/or in the claims), including for example when the same is/are incorporated into a system, apparatus, device and/or the like.

Various embodiments have been described with reference to specific example features thereof. It will, however, be evident that various modifications and changes may be made thereto without departing from the broader spirit and scope of the various embodiments as set forth in the appended claims. The specification and figures are, accordingly, to be regarded in an illustrative rather than a restrictive sense. It will be appreciated that, for clarity purposes, the above description has described embodiments with reference to different functional units. However, it will be apparent that any suitable distribution of functionality between different functional units may be used without detracting from the invention. For example, functionality illustrated to be performed by separate computing devices may be performed by the same computing device. Likewise, functionality illustrated to be performed by a single computing device may be distributed amongst several computing devices. Hence, references to specific functional units are only to be seen as references to suitable means for providing the described functionality, rather than indicative of a strict logical or physical structure or organization.

Although described above in terms of various example embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the present application, whether or not such embodiments are described and whether or not such features are presented as being a part of a described embodiment. Thus, the breadth and scope of the present application should not be limited by any of the above-described example embodiments.

Terms and phrases used in the present application, and variations thereof, unless otherwise expressly stated, should be construed as open ended as opposed to limiting. As examples of the foregoing: the term “including” should be read as meaning “including, without limitation” or the like; the term “example” is used to provide illustrative instances of the item in discussion, not an exhaustive or limiting list thereof; the terms “a” or “an” should be read as meaning “at least one,” “one or more” or the like; the term “set” should be read to include one or more objects of the type included in the set; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Similarly, the plural may in some cases be recognized as applicable to the singular and vice versa. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic, circuitry, or other components, may be combined in a single package or separately maintained and may further be distributed in multiple groupings or packages or across multiple locations.

Additionally, the various embodiments set forth herein are described in terms of example block diagrams, flow charts, and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration. Moreover, the operations and sub-operations of various methods described herein are not necessarily limited to the order described or shown in the figures, and one of skill in the art will appreciate, upon studying the present disclosure, variations of the order of the operations described herein that are within the spirit and scope of the disclosure. It will be understood that each block of the flowchart illustrations, and combinations of blocks in the flowchart illustrations, can be implemented by execution of computer program instructions. These computer program instructions may be loaded onto a computer or other programmable data processing apparatus (such as a controller, microcontroller, microprocessor or the like) in a sensor electronics system to produce a machine, such that the instructions which execute on the computer or other programmable data processing apparatus create instructions for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks presented herein.

It should be appreciated that all methods and processes disclosed herein may be used in any glucose or other analyte monitoring system, continuous or intermittent. It should further be appreciated that the implementation and/or execution of all methods and processes may be performed by any suitable devices or systems, whether local or remote. Further, any combination of devices or systems may be used to implement the present methods and processes.

In addition, the operations and sub-operations of methods described herein may be carried out or implemented, in some cases, by one or more of the components, elements, devices, modules, circuitry, processors, etc. of systems, apparatuses, devices, environments, and/or computing modules described herein and referenced in various of figures of the present disclosure, as well as one or more sub-components, elements, devices, modules, processors, circuitry, and the like depicted therein and/or described with respect thereto. In such instances, the description of the methods or aspects thereof may refer to a corresponding component, element, etc., but regardless of whether an explicit reference is made, one of skill in the art will recognize upon studying the present disclosure when the corresponding component, clement, etc. may be used. Further, it will be appreciated that such references do not necessarily limit the described methods to the particular component, element, etc. referred to. Thus, it will be appreciated by one of skill in the art that aspects and features described above in connection with (sub-) components, elements, devices, modules, and circuitry, etc., including variations thereof, may be applied to the various operations described in connection with methods described herein, and vice versa, without departing from the scope of the present disclosure.

1. An analyte sensor system, comprising:
an analyte sensor configured to generate analyte data associated with analyte levels of a user of the analyte sensor system;
an antenna system, comprising at least a first antenna and a second antenna, wherein:
the first antenna is configured to:
transmit, to a display device, a first signal including at least the analyte data; and
receive, from the display device, a second signal including operational instructions;
the second antenna is configured to:
receive the first signal from the first antenna and re-radiate the first signal towards the display device; and
receive the second signal from the display device and re-radiate the second signal towards the first antenna; and
a circuit board configured to operatively connect the analyte sensor with the first antenna of the antenna system.

2. The analyte sensor system of claim 1, wherein the first antenna and the circuit board are included within a housing of the analyte sensor system.

3. The analyte sensor system of claim 2, wherein the second antenna is included in the housing of the analyte sensor system.

4. The analyte sensor system of claim 2, wherein the second antenna is disposed outside of the housing of the analyte sensor system.

5. The analyte sensor system of claim 4, wherein the second antenna is incorporated into an adhesive patch attached to an outside of the housing of the analyte sensor system.

6. The analyte sensor system of claim 1, wherein:
the first antenna comprises a main antenna; and
the second antenna comprises a passive antenna.

7. (canceled)

8. The analyte sensor system of claim 6, wherein the passive antenna is integrated on the circuit board.

9. The analyte sensor system of claim 6, wherein the passive antenna comprises portions extending off the circuit board.

10. The analyte sensor system of claim 9, wherein the passive antenna extends to an outside portion of a housing of the analyte sensor system.

11. The analyte sensor system of claim 6, wherein:
the passive antenna comprises a passive antenna terminal; and
the passive antenna comprises one or more antenna arms coupled to the passive antenna terminal.

12. (canceled)

13. The analyte sensor system of claim 11, wherein the passive antenna is grounded at the passive antenna terminal.

14. The analyte sensor system of claim 11, wherein:
the passive antenna terminal comprises one or more passive electrical components;
the one or more passive electrical components comprise at least one of a resistor, a capacitor, or an inductor;
an electrical property of the one or more passive electrical components are based on at least one of a topology of the passive antenna or a distance from the passive antenna to the main antenna; and
the electrical property comprises at least one of resistance, capacitance, or inductance.

15-22. (canceled)

23. The analyte sensor system of claim 1, wherein at least one of:
the re-radiated first signal comprises a first same wavelength corresponding to the first signal; or
the re-radiated second signal comprises a second same wavelength corresponding to second signal.

24. The analyte sensor system of claim 1, wherein at least one of:
the second antenna is configured operatively such that the re-radiated first signal comprises a first different wavelength corresponding to the first signal; or
the second antenna is configured operatively such that the re-radiated second signal comprises a second different wavelength corresponding to the second signal.

25. The analyte sensor system of claim 1, wherein the second antenna is configured to modify a phase shift of at least one of the first signal or the second signal.

26. The analyte sensor system of claim 25, wherein at least one of:
a phase shift of the first signal re-radiated by the second antenna is different from a phase shift of the first signal received by the second antenna; or
a phase shift of the second signal re-radiated by the second antenna is different from a phase shift of the second signal received by the second antenna.

27. The analyte sensor system of claim 1, further comprising a transceiver, coupled to the first antenna, configured to:
transmit the first signal; and
receive at least one of the second signal from the display device or the re-radiated signal from the second antenna, wherein:
transceiver is configured to communicate signals, including the first signal, the second signal, and the re-radiated second signal, according to a wireless communication technology; and
the wireless communication technology comprises at least one of BLUETOOTH Low Energy (BLE), BLUETOOTH, or Wi-Fi.

28-29. (canceled)

30. The analyte sensor system of claim 1, further comprising one or more processors and one or more memories, wherein:
the circuit board is further configured to operatively connect the one or more processors and one or more memories to the analyte sensor, the first antenna, and a transceiver; and
the one or more processors are configured to:
obtain and process the analyte data from the analyte sensor; and
provide the processed analyte data to the transceiver for transmission via the first antenna.

31. (canceled)

32. An antenna system for communicating analyte data, comprising:
a first antenna operatively coupled to an analyte sensor via a circuit board, wherein the first antenna is configured to:
transmit, to a display device, a first signal including at least the analyte data; and
receive, from the display device, a second signal including operational instructions; and
a second antenna configured to:
receive the first signal from the first antenna and re-radiate the first signal towards the display device; and
receive the second signal from the display device and re-radiate the second signal towards the first antenna.

33-62. (canceled)

63. An analyte monitoring system, comprising:
a display device; and
an analyte sensor system comprising:
an analyte sensor configured to generate analyte data associated with analyte levels of a user of the analyte sensor system;
a first antenna configured to:
transmit, to the display device, a first signal including at least the analyte data; and
receive, from the display device, a second signal including operational instructions;
a second antenna configured to:
receive the first signal from the first antenna and re-radiate the first signal towards the display device; and
receive the second signal from the display device and re-radiate the second signal towards the first antenna; and
a circuit board configured to operatively connect the analyte sensor with the first antenna, wherein the display device is configured to:
receive the first signal including at least the analyte data;
display the analyte data received from the first antenna of the analyte sensor system to the user; and
transmit the second signal including the operational instructions.

64-149. (canceled)
